SEQUENCE LISTING (71 sequences)

SEQ ID NO: 1 - protein, 9 aa, Homo sapiens
SLYNTVATL

SEQ ID NO: 2 - protein, 34 aa, Homo sapiens
WMEWDREINNYTSLIHSLIEESQNQQEKNEQELL

SEQ ID NO: 3 - protein, 36 aa, Homo sapiens
SGIVQQQNNLLRAIEAQQHLLQLTVWGIKQLQARIL

SEQ ID NO: 4 - protein, 11 aa, Homo sapiens
RYLRDQQLLGI

SEQ ID NO: 5 - protein, 15 aa, Homo sapiens
PRGPDRPEGIEEEGG

SEQ ID NO: 6 - protein, 15 aa, Homo sapiens
GSNNNITLQCKIKQI

SEQ ID NO: 7 - protein, 15 aa, Homo sapiens
EGGERDRDTSGRLVH

SEQ ID NO: 8 - protein, 15 aa, Homo sapiens
KSQTQQEKNEQELLE

SEQ ID NO: 9 - protein, 15 aa, Homo sapiens
LGLLMICSATEKLWV

SEQ ID NO: 10 - protein, 15 aa, Homo sapiens
LFNSTWNGNNTWNNT

SEQ ID NO: 11 - protein, 15 aa, Homo sapiens
GTMLLGLLMICSATE

SEQ ID NO: 12 - protein, 15 aa, Homo sapiens
DTDTNDTEIFRPGGG

SEQ ID NO: 13 - protein, 15 aa, Homo sapiens
DRPEGIEEEGGERDR

SEQ ID NO: 14 - protein, 15 aa, Homo sapiens
NNNSNSEGTIKGGEM

SEQ ID NO: 15 - protein, 15 aa, Homo sapiens
DPNPQEVELVNVTEN

SEQ ID NO: 16 - protein, 15 aa, Homo sapiens
ASWSNKSLDDIWNNM

SEQ ID NO: 17 - protein, 15 aa, Homo sapiens
LKEQFKNKTIVFNQS

SEQ ID NO: 18 - protein, 15 aa, Homo sapiens
KWNDTLRQIVSKLKE

SEQ ID NO: 19 - protein, 15 aa, Homo sapiens
TVQCTHGIRPVVSTQ

SEQ ID NO: 20 - protein, 15 aa, Homo sapiens
IVELLGRRGWEVLKY

SEQ ID NO: 21 - protein, 15 aa, Homo sapiens
NYNKRKRIHIGPGRA

SEQ ID NO: 22 - protein, 15 aa, Homo sapiens
CTTTVPWNASWSNKS

SEQ ID NO: 23 - protein, 15 aa, Homo sapiens
NNTWNNTTGSNNNIT

SEQ ID NO: 24 - protein, 15 aa, Homo sapiens
AASVTLTVQARLLLS

SEQ ID NO: 25 - protein, 15 aa, Homo sapiens
GWEVLKYWWNLLQYW

SEQ ID NO: 26 - protein, 15 aa, Homo sapiens
LYKLDIVSIDNDSTS

SEQ ID NO: 27 - protein, 15 aa, Homo sapiens
LQARVLAVERYLKDQ

SEQ ID NO: 28 - protein, 15 aa, Homo sapiens
DKWASLWNWFDITNW

SEQ ID NO: 29 - protein, 15 aa, Homo sapiens
HMLQLTVWGIKQLQA

SEQ ID NO: 30 - protein, 15 aa, Homo sapiens
LLELDKWASLWNWFD

SEQ ID NO: 31 - protein, 15 aa, Homo sapiens
ISFEPIPIHYCAPAG

SEQ ID NO: 32 - protein, 15 aa, Homo sapiens
IWVDLRSLFLFSYHH

SEQ ID NO: 33 - protein, 15 aa, Homo sapiens
VITQACPKISFEPIP

SEQ ID NO: 34 - protein, 15 aa, Homo sapiens
KQIINMWQEVGKAMY

SEQ ID NO: 35 - protein, 9 aa, Homo sapiens
AMQMLKETI

SEQ ID NO: 36 - protein, 9 aa, Homo sapiens
RAHYNIVTF

SEQ ID NO: 37 - protein, 35 aa, Homo sapiens
GQAEPDRAHYNIVTFCCKCDSTLRLCVQSTHVDIR

SEQ ID NO: 38 - protein, 20 aa, Homo sapiens
GQAEPDRAHYNIVTFCCKCD

SEQ ID NO: 39 - protein, 8 aa, Homo sapiens
TLGIVCPI

SEQ ID NO: 40 - protein, 9 aa, Homo sapiens
FQQLFLNTL

SEQ ID NO: 41 - protein, 9 aa, Homo sapiens
FLLSLGIHL

SEQ ID NO: 42 - protein, 9 aa, Homo sapiens
CLTFGRETV

SEQ ID NO: 43 - protein, 9 aa, Homo sapiens
GLSRYVARL

SEQ ID NO: 44 - protein, 9 aa, Homo sapiens
ALMPLYACI

SEQ ID NO: 45 - protein, 9 aa, Homo sapiens
KLHLYSHPI

SEQ ID NO: 46 - protein, 10 aa, Homo sapiens
FLPSDFFPSV

SEQ ID NO: 47 - protein, 9 aa, Homo sapiens
FLLTRILTI

SEQ ID NO: 48 - protein, 9 aa, Homo sapiens
WLSLLVPFV

SEQ ID NO: 49 - protein, 9 aa, Homo sapiens
WLSLLVPFV

SEQ ID NO: 50 - protein, 9 aa, Homo sapiens
FLLTRILTI

SEQ ID NO: 51 - protein, 16 aa, Homo sapiens
WMNSTGFTKVCGAPPC

SEQ ID NO: 52 - protein, 16 aa, Homo sapiens
PYCWHYPPKPCGIVPA

SEQ ID NO: 53 - protein, 9 aa, Homo sapiens
  Amidation (9)..(9)
CDKCEARQE

SEQ ID NO: 54 - protein, 8 aa, Homo sapiens
SSIEFARL

SEQ ID NO: 55 - protein, 9 aa, Homo sapiens
IGITDRDFI

SEQ ID NO: 56 - protein, 9 aa, Homo sapiens
IGISNRDFV

SEQ ID NO: 57 - protein, 15 aa, Homo sapiens
QVYSLIRPNENPAHK

SEQ ID NO: 58 - protein, 10 aa, Homo sapiens
PACVYGPAVA

SEQ ID NO: 59 - protein, 17 aa, Homo sapiens
TDDSGHESDSNSNEGRH

SEQ ID NO: 60 - protein, 29 aa, Homo sapiens
YTIWMPENPRPGTPCDIFTNSRGKRASNG

SEQ ID NO: 61 - protein, 8 aa, Homo sapiens
PPPGIRGP

SEQ ID NO: 62 - protein, 10 aa, Homo sapiens
DWEYSVWLSN

SEQ ID NO: 63 - protein, 25 aa, Homo sapiens
AQGTLSKIFKLGGRDSRSGSPMARR

SEQ ID NO: 64 - protein, 20 aa, Homo sapiens
KYLATASTMDHARHGFLPRH

SEQ ID NO: 65 - protein, 25 aa, Homo sapiens
ASDYKSAHKGLKGVDAQGTLSKIFK

SEQ ID NO: 66 - protein, 20 aa, Homo sapiens
VTPRTPPPSQGKGRGLSLSR

SEQ ID NO: 67 - protein, 14 aa, Homo sapiens
YEYLINVIHAFQYV

SEQ ID NO: 68 - protein, 22 aa, Homo sapiens
VVHFFKNIVTPRTPPPSQGKGR

SEQ ID NO: 69 - protein, 14 aa, Homo sapiens
PGYPIRALVGDEAE

SEQ ID NO: 70 - protein, 15 aa, Homo sapiens
DEGGYTCFFRDHSYQ

SEQ ID NO: 71 - protein, 15 aa, Homo sapiens
SHLVEALYLVCGERG

(19) United States
(12) Patent Application Publication
(10) Pub. No.: US 2010/0151591 A1
(43) Pub. Date: Jun. 17, 2010

(54) RAPID HOMOGENEOUS DIAGNOSTIC TESTING PLATFORM BASED ON LANTHANIDE FLUORESCENT RESONANCE ENERGY TRANSFER

(76) Inventors: Nathaniel G. Butlin, San Francisco, CA (US); Larry Mimms, Poway, CA (US)

Correspondence Address: MORGAN, LEWIS & BOCKIUS LLP (SF), One Market, Spear Street Tower, Suite 2800, San Francisco, CA, 94105, US

(21) Appl. No.: 12/611,035

(22) Filed: Nov. 2, 2009

Related U.S. Application Data
(60) Provisional application No. 61/110,452, filed on Oct. 31, 2008.

(51) Int. Cl. G01N 33/53

(52) U.S. Cl. 436/501

(57) ABSTRACT

The present invention provides compositions and methods for detecting the presence of an analyte in a sample. Exemplary compositions comprise macrocyclic lanthanide complexes that are useful for transferring energy to an energy transfer acceptor to indicate the presence of the analyte. The compositions and methods are particularly suited for detecting antibodies in a sample.

### CROSS-REFERENCE TO RELATED APPLICATIONS

This application claims under 35 U.S.C. §119(e) the benefit of U.S. Application No. 61/110,452, filed Oct. 31, 2008, which is incorporated by reference in its entirety for all purposes.

### FIELD OF THE INVENTION

This invention relates to macrocyclic ligands and lanthanide complexes. Exemplary ligands or complexes are bound to probes for the detection of analytes, such as multivalent biomolecules.

### BACKGROUND OF THE INVENTION

A large number of mammalian disease states may be identified by the presence of circulating autologous antibodies, especially those specific to blood-born viruses such as Human Immunodeficiency Virus (HIV), Hepatitis A, B, C (HepA, HepB, HepC), Herpes Virus, and Human Papilloma Virus (HPV), as well as autoimmune diseases such as Rheumatoid Arthritis (RA), Multiple Sclerosis (MS), Lupus (Lp), and autoimmune thyroid diseases including Hashimoto's and Graves' Disease. Human infections are characterized by an immune response whereby the IgM antibodies are produced in the early stages of infection followed by the later generation of IgG antibodies that remain at elevated titers. Detection of these autologous antibodies (or IgA, IgE or IgD, for example) is an exceptional indication of past, recent, or present infection or diseased state. The need for a reliable and rapid diagnostic platform with significant environmental and temporal stability as well as ease and robustness of use, is significant and largely unmet in both the industrialized and developing world.

According to the UNAIDS 2008 Report on the global AIDS epidemic, at the end of 2007, approximately 33 million people were infected with HIV. Of the estimated 1.0 million people infected with HIV is the US, more than 25% are undiagnosed and it has been estimated that up to 30% of patients who test positive for HIV do not return to receive their results. A key aspect of HIV prevention is early and reliable, point of care (POC) diagnosis of infected individuals before they unknowingly put other individuals at risk. This is especially important in the developing world that shoulders 95% of the global HIV burden but lacks access to expensive analysis equipment and where rural patient populations and extreme environments render current tests inaccessible and unreliable. As technology advances slowly toward an effective vaccine, the need for a rapid, economical, robust, and portable diagnostic platform will become a crucial aspect of proving the effectiveness of any candidate vaccine. Current standard HIV testing methods include the Abbott Labs HIVAB EIA (that requires large, expensive, centrally located equipment and >90 minutes to complete) or the qualitative Oraquick Advance lateral flow test (that is expensive and difficult to use effectively in extreme environments) that requires visual interpretation of test results that can be highly variable.

There is thus a need for a point of care test that is stable and easy to use and interpret. The present invention addresses these and other problems.

### SUMMARY OF THE INVENTION

The invention provides in one aspect, a composition comprising: (a) a first probe comprising an energy transfer donor and a first targeting moiety; (b) a second probe comprising an energy transfer acceptor and a second targeting moiety; and (c) an antibody; wherein the energy transfer donor comprises a macrocyclic moiety complexed to a lanthanide ion, the macrocyclic moiety having a structure according to Formula I disclosed herein.

In another aspect, the invention provides a method of detecting an analyte in a sample comprising: (a) contacting the sample with (i) a first probe comprising an energy transfer donor and a first targeting moiety and (ii) a second probe comprising an energy transfer acceptor and a second targeting moiety to form an assay complex, wherein both the first probe and the second probe are bound to the analyte; and (b) detecting the presence of the assay complex.

### BRIEF DESCRIPTION OF THE DRAWINGS

FIG. 1 shows schematics of exemplary diagnostic tests. All representations of “peptides” in the figures can be substituted with any version of a larger protein containing a similar sequence with similar antigenic properties, and are not limited to viral protein origins.

FIG. 2 shows exemplary energy transfer donors useful in the invention. In FIG. 2, R is selected from a bond, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R links the energy transfer donor to a probe as described herein.

FIG. 3 shows A) Absorption (Excitation) and Emission spectrum of the compound of FIG. 2A. Multiple sharp emission peaks allow multiplexing of many independent tests in a single sample. B) Stability studies of the compound of FIG. 2A in varied buffers.

FIG. 4 shows A) Time resolved fluorescence reduces background emission by time-gated removal of short-lived biological fluorophores and proteins, lamp-glow, and autofluorescence of plastics and consumables. The compound of FIG. 2A has a 2.3 ms lifetime and is very stable toward repeated excitation allowing long delays and >1000 flashes. B) Example of reduced background achieved with time resolved fluorescence. Conventional fluorescence with fluorescein has linear range to ˜1.0 nM. Time resolved fluorescence of the compound of FIG. 2A gains more than 3 orders of magnitude sensitivity to <10 pM by gating out short-lived fluorescent interferences. All TRF signal charts shown herein are illustrative only, and the signal from any donor/acceptor combination will vary from assay to assay. Any indication of relative intensities is nothing more than illustrative, and should not be considered limiting.

FIG. 5 shows the effect of serum on Lumi4®-Tb time resolved fluorescence (TRF) signal.

FIG. 6 shows a heterogeneous label antigen binding experiment resulting in FRET signal.

FIG. 7 shows a dose response curve for a sample testing.

FIG. 8 shows results of an IgG detection assay on human serum.

### DETAILED DESCRIPTION OF THE INVENTION

### Definitions

“Analyte”, as used herein, means any compound or molecule of interest for which a diagnostic test is performed, such as a biopolymer or a small molecular bioactive material. An analyte can be, for example, a cell, a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, nucleic acid or lipid, without limitation.

As used herein, “energy transfer” refers to the process by which energy emission of an excited donor (e.g., a luminescent group) is altered by an acceptor (e.g., a luminescence-modifying group). When the luminescence-modifying group is a quenching group then the energy emission from the luminescent group is attenuated (quenched). Energy transfer mechanisms include luminescence resonance energy transfer, e.g., by dipole-dipole interaction (e.g., in longer range energy transfer) or electron transfer (e.g., across shorter distances). An exemplary mechanism involves transfer of energy from a metal chelate to a fluorophore (or a quencher or other luminescence modifying group) covalently bound to the chelating moiety through a linker, such as the compounds of the invention described herein. While energy transfer is often based on spectral overlap of the emission spectrum of the luminescent group and the absorption spectrum of the luminescence-modifying group, (in addition to distance between the groups) it has been demonstrated that spectral overlap is not necessarily required for energy transfer to occur (see, e.g., Latva et al., U.S. Pat. No. 5,998,146, which is incorporated herein by reference) and this type of energy transfer is encompassed within the present invention. Energy transfer between members of an energy transfer pair occurs when the members of the pair are in “operative proximity,” which is defined herein as a distance between the members of the pair that allows detectable energy transfer to occur. It is to be understood that any reference to “energy transfer” in the instant application encompasses all mechanistically-similar phenomena.

“Energy transfer pair” is used to refer to a group of molecules that participate in energy transfer. Such complexes may comprise, for example, two luminescent groups, which may be different from one another and one quenching group, two quenching groups and one luminescent group, or multiple luminescent groups and multiple quenching groups. In cases where there are multiple luminescent groups and/or multiple quenching groups, the individual groups may be different from one another. Typically, one of the molecules acts as a luminescent group, and another acts as a luminescence-modifying group. The preferred energy transfer pair of the invention comprises a luminescent group of the invention and a fluorophore (e.g., an organic fluorophore). The fluorophore can act as a quencher or other luminescence modifying group or, rather than a fluorophore, the acceptor-linker can be conjugated to a quencher or other luminescence modifying moiety. There is no limitation on the identity of the individual members of the energy transfer pair in this application. Generally preferred energy transfer pairs are characterized by a change in the spectroscopic properties of the pair if the distance between the individual members is altered by some critical amount. An exemplary energy transfer pair is a luminescent complex of the invention and an organic fluorophore. An energy transfer donor and an energy transfer acceptor may also compose an energy transfer pair.

As used herein, “luminescence-modifying group” refers to a molecule of the invention that can alter in any way the luminescence emission from a luminescent group. A luminescence-modifying group generally accomplishes this through an energy transfer mechanism. Depending on the identity of the luminescence-modifying group, the luminescence emission can undergo a number of alterations, including, but not limited to, attenuation, complete quenching, enhancement, a shift in wavelength, a shift in polarity, and a change in luminescence lifetime. One example of a luminescence-modifying group is a fluorophore that participates with a metal complex component of a complex of the invention in fluorescence resonance energy transfer. Another exemplary luminescence-modifying group is a quenching group.

As used herein, “quenching group” refers to any luminescence-modifying group of the invention that can attenuate at least partly the light emitted by a luminescent group. This attenuation is referred to herein as “quenching”. Hence, excitation of the luminescent group in the presence of the quenching group leads to an emission signal that is less intense than expected, or even completely absent. Quenching typically occurs through energy transfer between the luminescent group and the quenching group. In some instances, a quenching group may emit light upon relaxation. In others, the quenching group emits little or no light and hence may be referred to as a “dark quencher.”

“Fluorescence resonance energy transfer” or “FRET” is used interchangeably with and “LRET” and refers to an energy transfer phenomenon in which the excited state energy (e.g., light) emitted by an excited luminescent group is absorbed at least partially by a luminescence-modifying group of the invention and re-emitted at a different (e.g., longer) wavelength by the luminescence-modifying group. FRET depends on energy transfer between the luminescent group and the luminescence-modifying group. The efficiency of FRET depends at least in part on the distance between the luminescence modifying group and the luminescent group. In contrast to excimers and exciplex fluorescence, FRET pairs do not require the dye molecules forming the complexes to be in very close proximity. FRET is commonly used in several detection modes to detect, characterize or identify a variety of biologically active molecules including nucleic acids, e.g., oligonucleotides, peptides (e.g., peptides including one or more protease cleaveage site) and proteins (e.g., antibodies, antigens, receptors). One of the advantages of FRET is that fluorescence arises under physiologically relevant conditions (e.g., pH between about 7 and about 8, e.g., 7.3-7.5) in comparison to exciplex fluorescence which is typically weak under aqueous conditions, requiring the addition of organic solvents or formation in a similar molecular microenvironment. In one embodiment, the compound according to Formula I is incorporated into a nucleic acid having a motif of a known dual- or multiple-labeled nucleic acid probe (e.g., Molecular Beacons, Scorpion probes, TaqMan, and the like). The compound according to Formula I and the fluorophore can be positioned analogously to the donor and acceptor moieties of such probes.

“Moiety” refers to the radical of a molecule that is attached to another moiety.

The term “targeting moiety” is intended to mean any moiety of a compound, such as a probe, that targets the compound to a selected target (e.g., a complementary nucleic acid, a receptor structure, an antibody, an antigen, a lectin). The targeting moiety can be a small molecule (e.g., MW<500 D), which is intended to include both non-peptides and peptides. The targeting moiety can also be a macromolecule, which includes saccharides, lectins, receptors, ligands for receptors, proteins such as BSA, antibodies, nucleic acids, solid supports and so forth. The targeting moiety can be a component of a probe disclosed herein. In some embodiments, the targeting moiety is a probe. In another embodiment, the targeting moiety is a group conjugated to the functional moiety (e.g., a nucleic acid, antibody, antigen, biotin, avidin, streptavidin, etc.). One exemplary targeting moiety is an epitope that is recognized by an antibody. Another exemplary targeting moiety is an antigen or portion of an antigen that is recognized by an antibody. The targeting moiety will preferably bind to a target with high binding affinity. In other words, a targeting moiety with high binding affinity to a target has a high specificity for or specifically binds to the target. In some embodiments, a high binding affinity is given by a dissociation constant K_{d} of about 10⁻⁷M or less. In exemplary embodiments, a high binding affinity is given by a dissociation constant K_{d} of about 10⁻⁸ M or less, about 10⁻⁹ M or less, about 10⁻¹⁰ M or less, about 10⁻¹¹ M or less, about 10⁻¹²M or less, about 10⁻¹³ M or less, about 10⁻¹⁴M or less or about 10⁻¹⁵M or less. A compound may have a high binding affinity for a target if the compound comprises a targeting moiety that has a high binding affinity for the target.

Exemplary targets include multivalent biomolecules. The term “multivalent biomolecule” refers to a biomolecule having the ability to bind one or more copies of a binding partner, such as a probe as described herein. Examples of a multivalent biomolecule include a polypeptide, a nucleic acid, a lipid, a polysaccharide, a small molecule and derivatives thereof. One exemplary multivalent biomolecule is an antibody.

“Carrier moiety” or “carrier” as used herein refers to a species to which a compound according to Formula I is covalently bound through reaction of a reactive functional group on a functional moiety with a reactive functional group of complementary reactivity on the carrier moiety. Exemplary carrier moieties include nucleic acids (DNA, RNA), peptides, antibodies (such as IgG), antibody fragments, antigens, receptors, lectins, saccharides, lipids and the like. Further exemplary carriers include biotin, avidin and streptavidin. In some embodiments, the carrier moiety or carrier functions as a probe.

The term, “fluorophore,” as used herein refers to a species of that, when excited with light having a selected wavelength, emits light of a different wavelength. In some embodiments, a fluorophore has a structure other than that shown in Formula I or a luminescent metal complex of Formula I. Complexes of different metal ions incorporating the structure according to Formula I are considered to be different compounds. Thus, for example, if a Tb chelate is a complex according to Formula I, an identical Eu complex can be a “fluorophore” according to the present invention. A fluorophore can be covalently bound to a compound according to Formula I through a linker. Alternatively, the fluorophore can be bound to a first component of an assay, and the compound according to Formula I bound to a second component of an assay. Generally, it is preferred that the fluorophore be bound to the first assay component at a position and in a manner that allows energy transfer between the compound according to Formula I and the fluorophore when the first and second assay components interact in the assay. Other exemplary acceptors include quenchers and luminescence modifying moieties.

As used herein, “linker” refers to a moiety that links the chelating moiety of a compound of the invention to another species (e.g., carrier moiety or solid support). Exemplary linkers join a reactive functional group or a fluorophore to the chelating moiety of a compound of the invention. A linker can be any useful structure including, but not limited to 0-order linkers (i.e., a bond), acyl, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Further exemplary linkers include substituted or unsubstituted branched or linear C₁-C₁₀ substituted or unsubstituted alkyl and substituted or unsubstituted heteroalkyl. Other linkers include nucleic acids and peptides, such as PCR probes, hybridization probes and peptides that include protease cleavage sites. Still further linkers include antibodies, lectins, haptens and saccharides.

As used herein, “nucleic acid” means DNA, RNA, single-stranded, double-stranded, or more highly aggregated hybridization motifs, and any chemical modifications thereof. Exemplary modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Modifications can also include 3′ and 5′ modifications such as capping with a quencher, a fluorophore, an intercalator, a minor groove binder or another moiety. Exemplary nucleic acids will bind, preferably under stringent conditions, to a nucleic acid of diagnostic interest. Preferred nucleic acids of diagnostic interest are those that are correlated with a disease, condition or syndrome, or progression, amelioration or treatment of a disease, condition or syndrome. Nonlimiting examples of nucleic acids include those that are sufficiently complementary, to bind under stringent conditions, to a nucleic acid from hepatitis (e.g., A, B or C), human papilloma virus (HPV), human immunodeficiency virus (HIV), influenza, Severe Acute Respiratory Syndrome Virus (SARS), gram positive and gram negative bacteria, and antibiotic resistant bacterial infections, e.g., multiple resistant Staphylococcus (MRS).

“Peptide” refers to a homo- or hetero-polymer or -oligomer in which the monomers are amino acids and are joined together through amide bonds, alternatively referred to as a polypeptide. When the amino acids are alpha-amino acids, either the L-optical isomer or the D-optical isomer can be used. Additionally, unnatural amino acids, for example, beta.-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. The term “peptide” or “polypeptide”, as used herein, refers to naturally occurring as well as synthetic peptides. In addition, peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983). The “peptide,” “polypeptide” and “protein” may be used interchangeably.

An “antibody” refers a protein comprising one or more polypeptides substantially encoded by all or part of the recognized immunoglobulin genes. The recognized immunoglobulin genes, for example in humans, include the kappa (κ), lambda (λ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

“Reactive functional group,” as used herein, has the meaning generally recognized in the art of synthetic chemistry, particularly bioconjugate chemistry. Exemplary reactive functional groups included, without limitation, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Methods to prepare each of these functional groups are well-known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

Where substituent groups are specified by their conventional chemical formulae, written from left to right, they equally encompass the chemically identical substituents, which would result from writing the structure from right to left, e.g., —CH₂O— is intended to also recite —OCH₂—. Thus, the incorporation of a moiety depicted with two attachment points into a larger structure is not limited to the depicted orientation of the moiety.

The term “alkyl,” by itself or as part of another substituent, means, unless otherwise stated, a straight or branched chain, or cyclic hydrocarbon radical, or combination thereof, which may be fully saturated, mono- or polyunsaturated and includes mono-, di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds (i.e., alkenyl and alkynyl moieties). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl and heteroalkyl groups attached to the remainder of the molecule via an oxygen atom, a nitrogen atom (e.g., an amine group), or a sulfur atom, respectively.

The term “heteroalkyl,” by itself or in combination with another term, means, unless otherwise stated, a stable straight or branched chain, or cyclic alkyl moiety, or combinations thereof, consisting of a number (e.g., a stated number) of carbon atoms and at least one heteroatom selected from the group consisting of O, N, Si, B and S, and wherein the nitrogen and sulfur atoms may optionally be oxidized and the nitrogen heteroatom may optionally be quaternized. The heteroatom(s) O, N, S, B and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “acyl” refers to a species that includes the moiety —C(O)R, where R has the meaning defined herein.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “cycloalkyl,” “heterocycloalkyl,” “aryl,” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ′NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted heterocycloalkyl, substituted or unsubstituted aryl, substituted or unsubstituted heteroaryl, halogen, —OR′, ═O, ═NR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), silicon (Si) and boron (B).

A “linkage fragment” is formed by reaction of a reactive functional group on one species with reactive functional group of complementary reactivity on another species (e.g., a fluorophore and an acceptor-linker, a functional moiety and a carrier moiety (or solid support). Exemplary linkage fragments formed by such reactions include, but are not limited to S, SC(O)NH, SC(O)(NH)₂, HNC(O)S, SC(O)O, O, NH, NHC(O), (NH)₂C(O), (O)CNH and NHC(O)O, and OC(O)NH, CH₂S, CH₂O, CH₂CH₂O, CH₂CH₂S, (CH₂)ₚO, (CH₂)ₚS or (CH₂)ₚY′-PEG wherein, Y′ is S, NH, NHC(O), C(O)NH, NHC(O)O, OC(O)NH, or O and p is an integer from 1 to 50.

In some embodiments, the symbol “R” is a general abbreviation that represents a substituent group that is selected from a bond, acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

The present invention includes all salt forms of those molecules that contain ionizable functional groups, such as basic and acidic groups.

When a residue (such as “R”) is defined herein as a single negative charge, then the residue can include a cationic counterion. The resulting salt form of the compound is encompassed in the structure as presented.

### INTRODUCTION

The invention provides compositions and methods for the detection of an analyte in a sample based on the transfer of energy from one component of the compositions to another component. While a number of different energy transfer mechanisms may be employed, preferred mechanisms include fluorescence resonance energy transfer. One advantage of the approach taken herein is that in some embodiments, the methods are fundamentally different from traditional enzyme immunoassays (EIA) in that they require no wash steps and are therefore truly homogeneous assays. In the simplest manifestation of this test, a lyophilized pellet containing the labeled probes is added to the patient specimen. After a short incubation time, fluorescent signal is measured in a hand held detector.

In one aspect, the invention provides a composition comprising a first probe comprising an energy transfer donor and a first targeting moiety; a second probe comprising an energy transfer acceptor and a second targeting moiety; and a multivalent biomolecule. Generally, the first targeting moiety and second targeting moiety are portions of the first and second probe, respectively, that are each characterized by a high binding affinity for the multivalent biomolecule. As discussed below, binding of the first probe and the second probe to the multivalent biomolecule brings the energy transfer donor and the energy transfer acceptor in sufficiently close proximity (“operative proximity”) so as to allow the detectable transfer of energy between them. The distance separating the energy transfer donor and acceptor at which energy transfer can be detected varies according to the specific mechanism used. For example, in FRET, distances of about 10 nm may represent the outer limits of efficient transfer between donor and acceptor. Accordingly, in one embodiment, the first probe, the second probe or both are bound to the multivalent biomolecule.

The invention provides probes suitable for the methods and compositions disclosed herein. The probes can be any molecule or aggregation of molecules whose binding to a multivalent biomolecule allows for the detection of the multivalent biomolecule. Useful probes include polypeptides, nucleic acids, lipids, polysaccharides, small organic molecules (e.g., MW<500 D), hormones, glycogens or derivatives thereof. In exemplary embodiments, the probe comprises a moiety that can be bound by circulating antibodies. The probes may be in a highly purified state but may also be part of an unpurified mixture such as a cell lysate, a population of various length peptides of some sequence, or a mixture of multiple full length peptides that might be more reactive to the overall polyclonal nature of an immune response. A probe generally comprises one of an energy transfer pair (i.e., either an energy transfer donor or an energy transfer acceptor) and a targeting moiety. In exemplary embodiments, a probe is polypeptide. In some embodiments, a probe is an antibody. In some embodiments, a probe is an antigen or portion of an antigen (such as an epitope).

In exemplary embodiments, a first probe comprises an energy transfer donor and a first targeting moiety and a second probe comprises an energy transfer acceptor and a second targeting moiety, wherein the first targeting moiety and the second targeting moiety have substantially the same structure. In exemplary embodiments, a first probe comprises an energy transfer donor and a first targeting moiety and a second probe comprises an energy transfer acceptor and a second targeting moiety, wherein the first probe and the second probe have substantially the same structure. By “substantially the same structure” is meant that each of two probes or each of two targeting moieties binds the same binding partner with similar specificity (e.g., their K_{d} differs by no more than 10%). When two probes are nucleic acids or polypeptides, “substantially the same structure” can mean that the two probes or their targeting moieties share a homology that is equal to or greater than about a percentage selected from 80%, 90%, 91%, 92%, 93%, 94%, 95%, 96%, 97%, 98% and 99%, preferably selected from 95%, 96%, 97%, 98% and 99%, more preferably 99%. In some embodiments, two probes or two targeting moieties have substantially the same structure if one of the probes or targeting moieties competes with the other of the probes or targeting moieties for binding to a binding partner. In exemplary embodiments, two probes have substantially the same structure if their targeting moieties are epitopes to a same antibody. In exemplary embodiments, two targeting moieties have substantially the same structure if they are epitopes to a same antibody. In some embodiments, two or more targeting moieties or probes of the composition do not have substantially the same structure.

In an exemplary embodiment, the multivalent biomolecule is an antibody.

In an exemplary embodiment, the multivalent biomolecule is an antibody that specifically binds to a polypeptide. In an exemplary embodiment, the multivalent biomolecule is an antibody that specifically binds to a polypeptide associated with a disease causing agent. In an exemplary embodiment, the multivalent biomolecule is an antibody that specifically binds to a polypeptide associated with a virus (for example, Human Immunodeficiency Virus (e.g., HIV-1 and HIV-2); Hepatitis (e.g., HepA, HepB, HepC and HepG); Herpes Simplex; Yellow Fever; Dengue 2; Influenza; Epstein-Barr; Rabies; and Human Papilloma Virus (HPV)). In an exemplary embodiment, the multivalent biomolecule is an antibody that specifically binds to a polypeptide associated with an autoimmune disease (for example, Lupus Erythematosus and Multiple Sclerosis). In an exemplary embodiment, the multivalent biomolecule is an antibody that specifically binds to a polypeptide associated with diabetes. Thus, in any of these embodiments, the multivalent biomolecule is an antibody whose presence is detected by binding of a first polypeptide probe comprising an energy transfer donor and a first targeting moiety and a second polypeptide probe comprising an energy transfer acceptor and a second targeting moiety.

In some embodiments, a probe that may be used to bind to the multivalent biomolecule (such as an antibody) is a polypeptide comprising a sequence selected from the following table:

| | Sequence | Amino Acid |
|---|---|---|
| Disease | Name | Sequence |
| Human | SL9, p17 (77- | SLYNTVATL |
| Immunodeficiency | 85) | |
| Virus (HIV-1) | | |
| | C34, gp41 | WMEWDREINNYTSLIHSL |
| | Fragment | IEESQNQQEKNEQELL |
| | N36, HR1, | SGIVQQQNNLLRAIEAQQ |
| | gp41 | HLLQLTVWGIKQLQARIL |
| | Fragment | |
| | env (584-594) | RYLRDQQLLGI |
| | MN ENV-182 | PRGPDRPEGIEEEGG |
| | MN ENV-103 | GSNNNITLQCKIKQI |
| | MN ENV-185 | EGGERDRDTSGPLVH |
| | MN ENV-163 | KSQTQQEKNEQELLE |
| | MNENV-6 | LGLLMICSATEKLWV |
| | MN ENV-99 | LFNSTWNGNNTWNNT |
| | MN ENV-5 | GTMLLGLLMICSATE |
| | MN ENV-116 | DTDTNDTEIFRPGGG |
| | MN ENV-183 | DRPEGIEEEGGERDR |
| | MN ENV-37 | NNNSNSEGTIKGGEM |
| | MN ENV-20 | DPNPQEVELVNVTEN |
| | MN ENV-154 | ASWSNKSLDDIWNNM |
| | MN ENV-89 | LKEQFKNKTIVFNQS |
| | MN ENV-86 | KWNDTLRQIVSKLKE |
| | MN ENV-63 | TVQCTHGIRPVVSTQ |
| | MN ENV-166 | IVELLGRRGWEVLKY |
| | MN ENV-77 | NYNKRKRIHIGPGRA |
| | MN ENV-152 | CTTTVPWNASWSNKS |
| | MN ENV-101 | NNTWNNTTGSNNNTT |
| | MN ENV-134 | AASVTLTVQARLLLS |
| | MN ENV-1987 | GWEVLKYWWNLLQYW |
| | MNENV-46 | LYKLDTVSTDNDSTS |
| | MN ENV-145 | LQARVLAVERYLKDQ |
| | MN ENV-167 | DKWASLWNWFDTTNW |
| | MN ENV-142 | HMLQLTVWGTKQLQA |
| | MN ENV-166 | LLELDKWASLWNWFD |
| | MNENV-54 | ISFEPIPIHYCAPAG |
| | MN ENV-190 | IWVDLRSLFLFSYHH |
| | MNENV-52 | VITQACPKISFEPIP |
| | MN ENV-106 | KQIINMWQEVGKAMY |
| | HIV gag p24 | AMQMLKETT |
| | peptide (197- | |
| | 205) | |
| | Gag p24 | Any derivative |
| | protein | sequence |
| | p41 protein | Any derivative |
| | | sequence |
| Human | p36 protein | Any derivative |
| Immunodeficiency | | sequence |
| Virus (HIV-2) | | |
| Human Papilloma | E7 protein | RAHYNIVTF |
| Virus (HPV) | (49-57) | |
| | E7 (43-77), | GQAEPDRAHYNIVTFCCK |
| | HPV | CDSTLRLCVQSTHVDTR |
| | Oncoprotein | |
| | E7 (43-62), | GQAEPDRAHYNIVTFCCK |
| | HPV | CD |
| | Oncoprotein | |
| | HPV16 E7(86- | TLGIVCPI |
| | 93) | |
| | (HPV18) E7 | FQQLFLNTL |
| | protein (86- | |
| | 94) | |
| Hepatitis B Virus | HBVpol575, | FLLSLGIHL |
| | HBV | |
| | polymerase | |
| | (575-583) | |
| | HBV Core | CLTFGRETV |
| | (107-115) | |
| | HBVpol455 | GLSRYVARL |
| | HBV | |
| | polymerase | |
| | (455-463) | |
| | HBVpol655, | ALMPLYACI |
| | HBV | |
| | polymerase | |
| | (655-663) | |
| | HBVpol502, | KLHLYSHPI |
| | HBV | |
| | polymerase | |
| | (502-510) | |
| | HBcAg | FLPSDFFPSV |
| | (HBV)(18- | |
| | 27) | |
| | HBVenv | FLLTRILTI |
| | (183-191) | |
| | HBVenv (335- | WLSLLVPFV |
| | 343) | |
| | HBVenv (335- | WLSLLVPFV |
| | 343) | |
| | HBVenv | FLLTRILTI |
| | (183-191) | |
| Hepatitis C Virus | HCV-1 e2 | WMNSTGFTKVCGAPPC |
| | Protein (554- | |
| | 569) | |
| | HCV-1 e2 | PYCWHYPPKPCGIVPA |
| | Protein (484- | |
| | 499) | |
| Hepatitis G Virus | HGV (2268- | CDKCEARQE-NH₂ |
| | 2276) | |
| Herpes Simplex | HSV-gB2 | SSIEFARL |
| | (498-505) | |
| Yellow Fever | Virus | IGITDRDFI |
| | Envelope | |
| | Protein E1 | |
| Dengue 2 | Virus | IGISNRDFV |
| | Fragment | |
| Influenza | Nucleoprotein | QVYSLIRPNENPAHK |
| | (311-325) | |
| | Nucleoprotein | PACVYGPAVA |
| | (277-286) | |
| Epstein-Barr | EBV, | TDDSGHESDSNSNEGRH |
| | LMP1, TDD | |
| Rabies | Virus | YTIWMPENPRPGTPCDIF |
| | Glycoprotein | TNSRGKRASNG |
| | (RVG) | |

Autoimmune diseases

| | | |
|---|---|---|
| Lupus | Epitope 1 | PPPGIRGP |
| Erythematosus | | |
| | dsDNA | DWEYSVWLSN |
| | Mimetope | |
| | Peptide | |
| Multiple Sclerosis | Myelin Basic | AQGTLSKIFKLGGRDSRS |
| | Protein | GSPMARR |
| | (MBP) (146-170 | |
| | MBP (14-33) | KYLATASTMDHARHGFLP |
| | MBP (131-155) | ASDYKSAHKGLKGVDAQG |
| | MBP (92-111) | VTPRTPPPSQGKGRGLSL |
| | | SR |
| | PLP (57-70) | YEYLINVIHAFQYV |
| | MBP (84-105) | VVHFFKNIVTPRTPPPSQ |
| | | GKGR |
| | MOG (8-21) | PGYPIRALVGDEAE |
| | MOG (92-106 | DEGGYTCFFRDHSYQ |

Other Diseases

| | | |
|---|---|---|
| Type 1 Diabetes | Insulin B (9- | SHLVEALYLVCGERG |
| | 23) | |

The compositions and methods of the invention are widely applicable to all disease and infections states in which the host organism generates an immune response. Broad examples of these areas are blood viral infections and autoimmune disorders. The table above lists some non-limiting exemplary disease states and associated peptidic probes. In many cases, a short peptidic probe is derived from a protein probe, and it should be understood that the compositions and methods of the invention are equally viable utilizing a protein probe in place of the elaborated peptidic probe. Specifically, any amino acid sequence that is derived from the overall sequence of either a mature protein or its DNA coding sequence may be leveraged for use in the current invention. Additionally, synthetic constructs are equally viable for use in the present invention and may take the form of truncated derivative, fusion proteins of two naturally occurring proteins, or a natural protein and a synthetic tag or sequence, and non-native mutations as non-limiting examples.

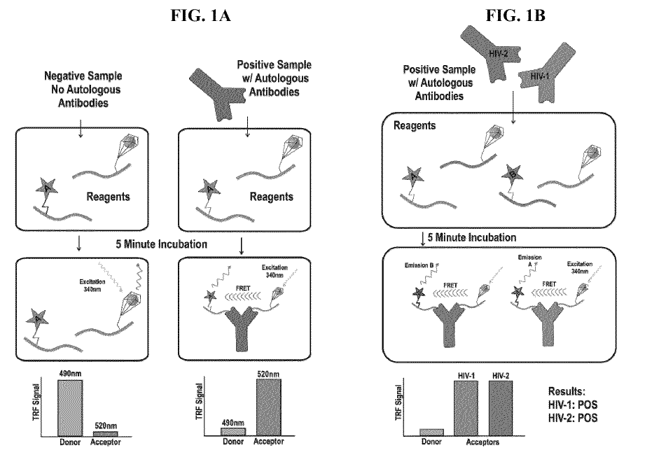

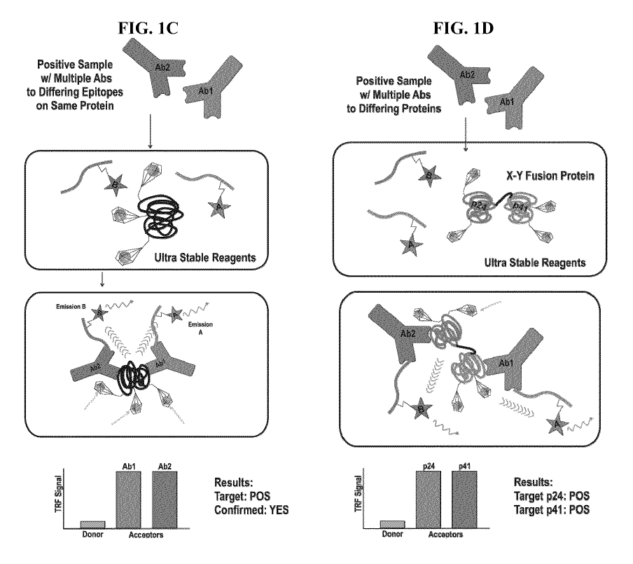

The probes disclosed herein may be bound to multivalent biomolecules in a wide variety of formats. FIG. 1 shows one example, in which a first probe comprises a fluorophore donor and a second probe comprises a fluorophore acceptor. The first probe and the second probe both can bind to a target multivalent biomolecule, which in this case is an antibody. On the left side of FIG. 1A, energy transfer between the fluorophore donor and the fluorophore acceptor is not detectable because of the absence of the antibody. On the right side of FIG. 1A, binding of the first probe and the second probe brings the fluorophore donor and the fluorophore acceptor into close proximity. As a result, FRET transfer can occur, and the TRF signal of the acceptor is increased.

In another format, the compositions of the invention can be used to detect the presence of a plurality of multivalent biomolecules. For example, it can be desirable to identify related circulating autologous antibody varieties such as HIV-1 and HIV-2, or HIV-1 sub-types A-K from within a multiplexed format. This is outlined in FIG. 1B for the HIV-1 and HIV-2 virus types. The multiplex potential of this invention is limited only by the number of acceptor emission that can be effectively discriminated and the cross-reactivity of the circulating antibodies generate by the host. As described herein, a multiplexed test would differ from the monoplex test in that two sets of probes may be assembled together and that the acceptors for each test are spectroscopically discernable. For example, Acceptor A below may be fluorescein with an emission wavelength of 520 nm and acceptor B may be Cy₅ with an emission wavelength of 670 nm.

Accordingly, in one aspect, the invention provides a composition comprising a first probe comprising a first energy transfer donor and a first targeting moiety; a second probe comprising a first energy transfer acceptor and a second targeting moiety; a third probe comprising a second energy transfer donor and a third targeting moiety; a fourth probe comprising a second energy transfer acceptor and a fourth targeting moiety; a first multivalent biomolecule and a second multivalent biomolecule. In exemplary embodiments, the first targeting moiety and the second targeting moiety have substantially the same structure, and the third targeting moiety and the fourth targeting moiety have substantially the same structure. In exemplary embodiments, the first probe and the second probe are bound to the first multivalent biomolecule and the third probe and the fourth probe are bound to the second multivalent biomolecule. As can be appreciated by one of skill in the art, additional multivalent biomolecules can be added and detected with the addition of additional probes that are used to generate a distinct signal.

In another aspect, the invention provides a composition comprising a first probe comprising a first energy transfer donor, a first targeting moiety and a second targeting moiety; a second probe comprising a first energy transfer acceptor and a third targeting moiety; a third probe comprising a second energy transfer acceptor and a fourth targeting moiety; a first multivalent biomolecule and a second multivalent biomolecule. In exemplary embodiments, the first targeting moiety and the second targeting moiety do not have substantially the same structure. In exemplary embodiments, the first targeting moiety and the second targeting moiety do not have substantially the same structure; the first targeting moiety and the third targeting moiety have substantially the same structure; and the second targeting moiety and the fourth targeting moiety have substantially the same structure. In exemplary embodiments, the first probe and the second probe are bound to the first multivalent biomolecule, and the first probe and the third probe are bound to the second multivalent biomolecule.

One example is shown in FIG. 1C. Here, a combination as described above is used in a “confirmatory” mode whereby the polyclonal nature of the circulating antibodies is leveraged for the assessment of a particular epitope and further confirmed via the presence of a second peptide epitope (FIG. 1C). In this non-limiting example, the combination comprises a whole or partial protein and a plurality of multiple peptides, all derived from the protein. Peptides are labeled with either donor or acceptor fluorophores which are different for each sequence and the protein is labeled with an appropriate donor or acceptor. Multiple polyclonal antibodies bind differing epitopes on the protein structure in one arm and analogous peptides in the other arm. When a sample contains antibodies to both epitopes of the same protein, they bring both donor and acceptor fluors into close proximity and FRET signal is observed. The confirmatory wavelength serves to provide additional evidence of the presence of antibodies to a single protein in circulation. There is expectation that the described arrangement of a macromolecule that is multiply labeled with donor fluors will offer significant signal enhancement for both wavelengths once the larger FRET transfer structure is assembled.

Another example is depicted in FIG. 1D, which shows an additional format of interest that includes an engineered fusion protein that contain at least 2 recognizable epitopes from at least 2 unique proteins, each representing a unique pathogenic or diseased state. In this format, a complex is made with multiple acceptor labeled peptides and multiply circulating antibodies around a central fusion protein such that protein targets can represent completely different disease states, and the test will be able to and reliably readout a result for each from a single sample. In FIG. 1D, a fusion protein between the p24 and p41 antigens of HIV-1 is indicated and represents two unique proteins from the same disease state, HIV infection. One of skill in the art can appreciate that these two proteins could represent different disease states in a multiplex assay such Hepatitis A and Hepatitis B or HepA and HIV-1.

In another format, the compositions of the invention can be used in a competitive format. Here, a first multivalent biomolecule is unlabeled, while a second multivalent biomolecule is labeled with either an energy transfer donor or acceptor. The increased presence of the first multivalent biomolecule will reduce the overall assay signal by occupying binding sites for donor or acceptor molecules.

### Energy Transfer Donors

A variety of energy transfer donors known to those of skill in the art can be utilized in the invention. In an exemplary embodiment, the energy transfer donor is a donor fluorophore. In an exemplary embodiment, the energy transfer donor comprises a macrocyclic fluorescent complex. In an exemplary embodiment, the energy transfer donor comprises a macrocyclic moiety complexed to a lanthanide ion.

In an exemplary embodiment, the energy transfer donor comprises a macrocyclic moiety complexed to a lanthanide ion, the macrocyclic moiety having a structure according to Formula I:

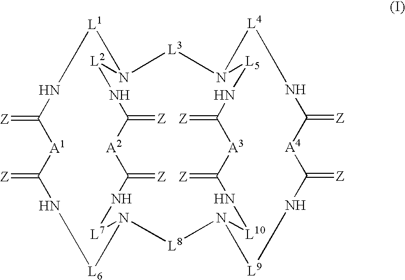

In Formula (I), each Z is a member independently selected from O and S. L¹, L², L³, L⁴, L⁵, L⁶, L⁷, L⁸, L⁹ and L¹⁰ (“L^{x}” moieties) are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. A¹, A², A³ and A⁴ are members independently selected from the general structure:

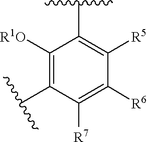

wherein each R¹ is a member independently selected from H, an enzymatically labile group, a hydrolytically labile group, a metabolically labile group and a single negative charge. Each R⁵, R⁶ and R⁷ (“an R^{x} moiety”) is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂. R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl. R¹⁷ and R¹⁸ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁷ and R¹⁸, together with the atoms to which they are attached, are optionally joined to form a 5- to 7-membered ring.

In an exemplary embodiment, an energy transfer donor according to Formula I is covalently attached through a functional moiety to a probe.

Exemplary energy transfer donors according to any of the embodiments discussed herein include those in which at least one L^{x} or R^{x} moiety is functionalized with a linker and is bound to the probe through a linkage fragment. In one embodiment, at least one L^{x} or R^{x} moiety is functionalized with a functional moiety. In yet another exemplary embodiment, one of these L^{x} or R^{x} groups is functionalized with a linker and the same or a different L^{x} or R^{x} moiety is functionalized with a functional moiety.

Thus, the present invention provides energy transfer donors according to Formula I in which at least one L^{x} moiety is substituted with a group selected from:

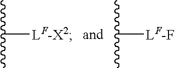

in which L^{F} is a linker as described herein, and X² is a reactive functional group. F is a probe bound to L^{F} through a linkage fragment formed as described herein.

In another embodiment, the invention provides compounds according to Formula I in which at least one L^{x} moiety is substituted with a group selected from:

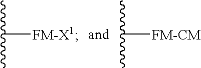

in which FM is a functional moiety as described herein, having as a component a reactive functional group, X¹. CM is a carrier moiety (or solid support) bound to FM through a linkage fragment formed as described herein.

In still a further embodiment, the invention provides a compound according to Formula I in which at least one L^{x} moiety is substituted with a group selected from:

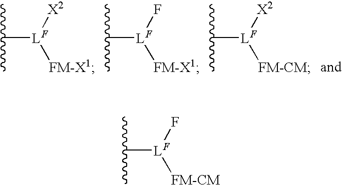

in which the moieties are as described above. As will be appreciated by those of skill in the art, the linker L^{F} can be conjugated to one or more quencher or other luminescence modifying moiety.

In general, the metal complex will serve as a donor fluorophore, and will have a longer excited state lifetime than an acceptor fluorophore. In an exemplary embodiment, the donor fluorophore is a lanthanide chelate. In another exemplary embodiment, the acceptor fluorophore is an organic fluorophore, e.g., a polyaromatic hydrocarbon (e.g., a hetrocyclic compound).

Transfer of excited state energy from a donor fluorophore to an acceptor fluorophore provides an acceptor fluorophore with a longer excited state lifetime than an identical fluorophore that is not excited by the donor. The acceptor fluorophore generally luminesces at a wavelength longer than that of the energy incoming from the donor.

In another exemplary embodiment, the compositions of the invention include multiple donor fluorophores. In a further embodiment, the compositions of the invention include multiple acceptor fluorophores. The compositions can include both multiple donor and multiple acceptor fluorophores (or quenchers or other luminescence modifying moieties).

The luminescent complexes according to Formula I, in conjunction (e.g., operative contact allowing exchange of energy) with energy transfer to a fluorophore, provides a luminescent system that is tunable with respect to emission wavelength. The emission wavelength is tunable because, when energy transfer is chosen to be large, emission color is principally determined by the emission wavelength of the fluorophore, which can be selected for its output color.

The complexes in conjunction with the fluorophore are also tunable with respect to emission lifetime because the lifetime is determined by the efficiency of energy transfer from the complex of Formula Ito the fluorophore. The fluorophore typically has a short lifetime. Because it is continuously excited by the luminescent complex of Formula I, its emission intensity decays with a lifetime related to the lifetime of the luminescent complex. The lifetime can be tuned by altering the distance between the luminescent complex and the fluorophore. The Foerster equation is of use to predict the lifetime of the energy transfer pair.

In another exemplary embodiment, the energy transfer donor has the structure:

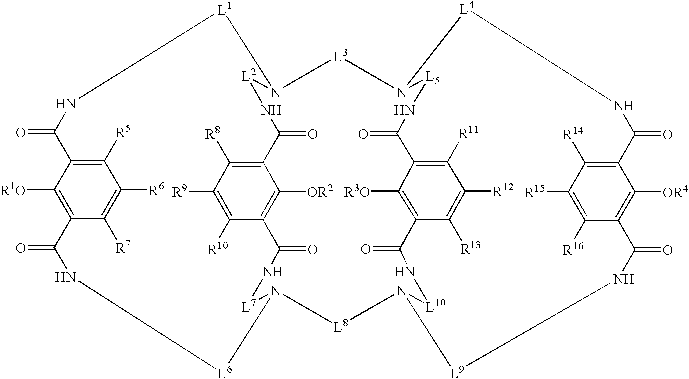

wherein R¹, R², R³ and R⁴ are members independently selected from H, an enzymatically labile group, a hydrolytically labile group, a metabolically labile group, a group that is cleaved by incident light and a single negative charge. The substituents and attributes of compounds according to this embodiment are as described above with reference to Formula I. Any one or more than one of L¹, L², L³, L⁴, L⁵, L⁶, L⁷, L⁸, L⁹ and L¹⁰ can be substituted with one or more functional moiety and/or linker.

In another exemplary embodiment, the linker moieties L¹, L², L³, L⁴, L⁵, L⁶, L⁷, L⁸, L⁹ and L¹⁰ are members independently selected from substituted or unsubstituted C₁ to C₆ alkyl. Exemplary compounds include those in which L¹, L², L³, L⁴, L⁵, L⁶, L⁷, L⁸, L⁹ and L¹⁰ are members independently selected from substituted or unsubstituted ethyl.

An exemplary ligand according to this embodiment has the structure:

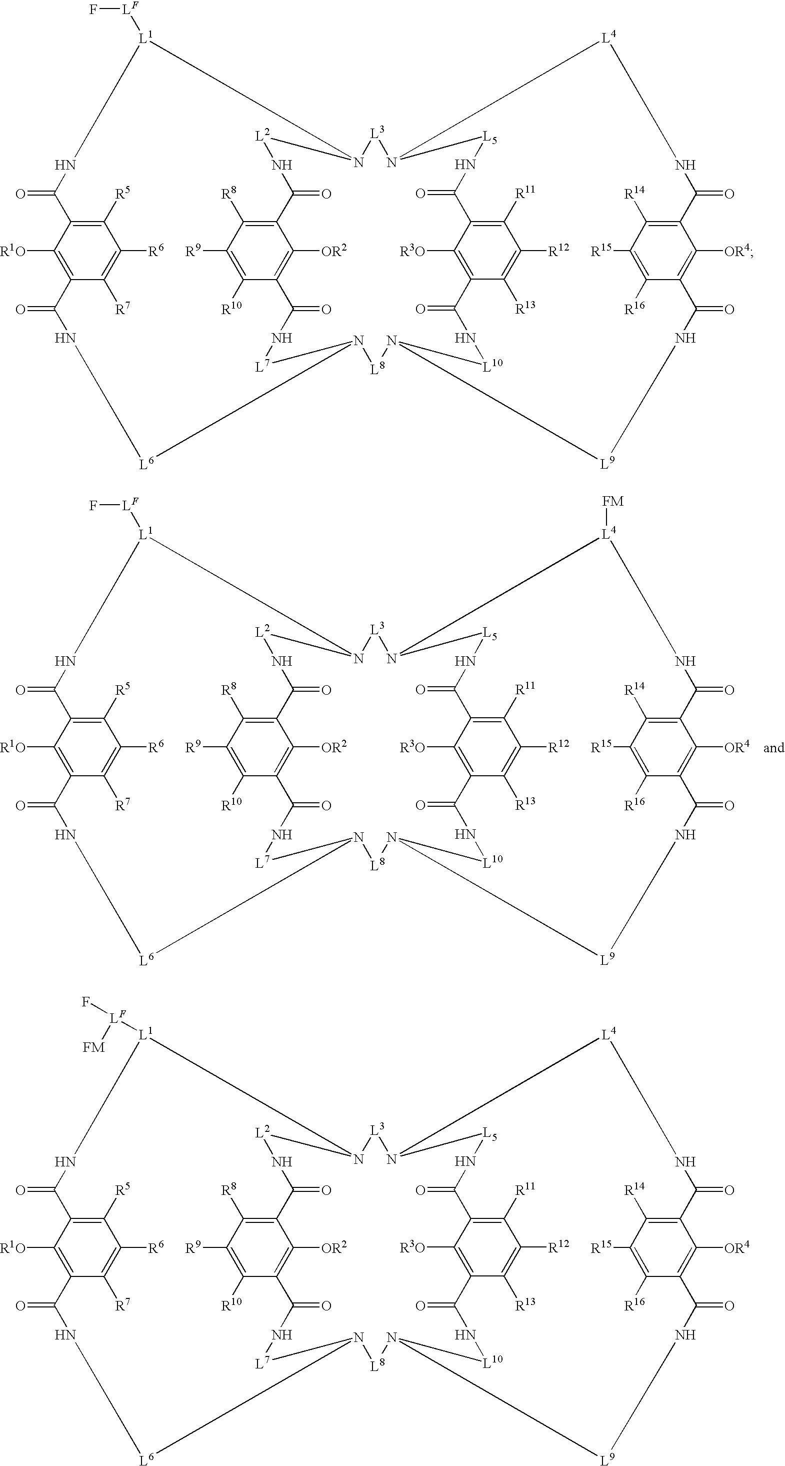

wherein L^{F} is a linker, F represents a probe and FM is a functional moiety. As will be appreciated by those of skill, the linker and functional moiety can be attached to any one or more than one of L¹, L², L³, L⁴, L⁵, L⁶, L⁷, L⁸, L⁹ and L¹⁰.

In another embodiment, the compound of the invention has a formula selected from:

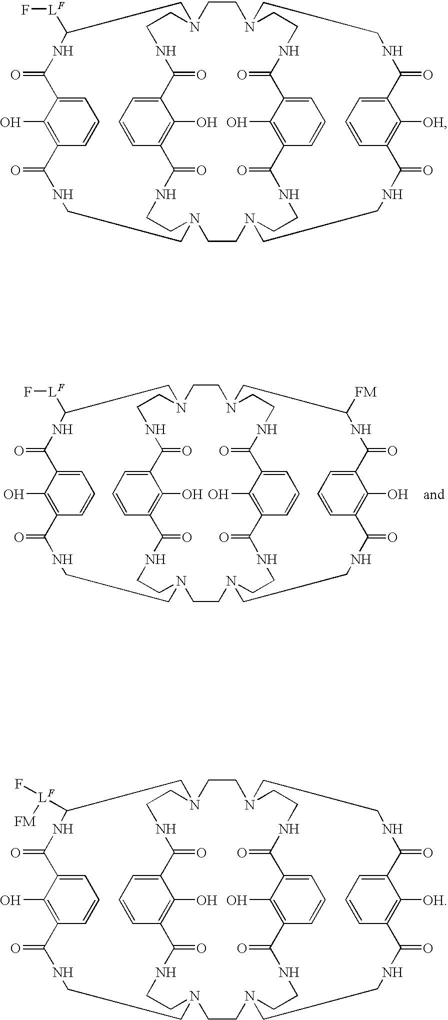

Each of the structures above is intended to include those derivatives in which the probe linker is not conjugated to a probe as well as those conjugated to a probe. Also included are those derivatives in which the functional moiety is conjugated to a carrier moiety (CM) as well as those that are conjugated to a carrier moiety or solid support.

In another exemplary embodiment, the invention provides a compound having the formula:

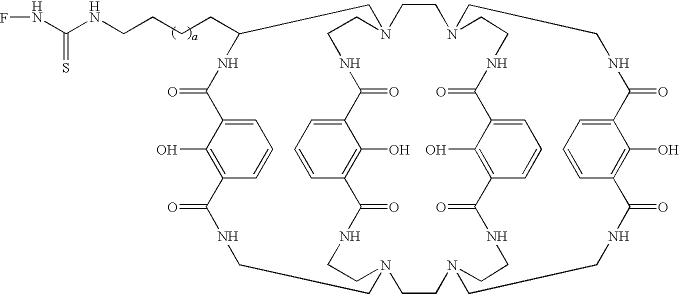

in which a is an integer greater than or equal to 0, e.g., from 0 to 10.

In another exemplary embodiment, the compound of the invention includes an amide linkage, which is more stable than the thiourea:

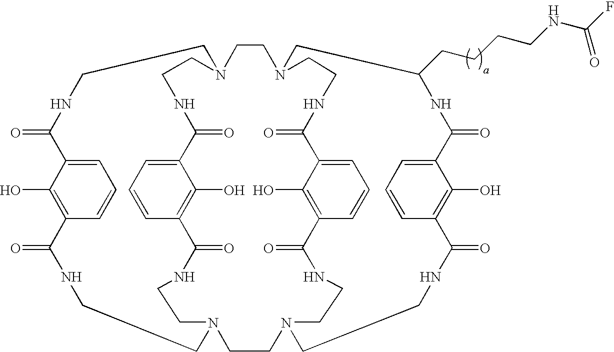

Compounds of the inventions including both a probe linker moiety, optionally attached to a probe, and a functional moiety, optionally attached to a carrier moiety (or solid support) are exemplified by the following compounds:

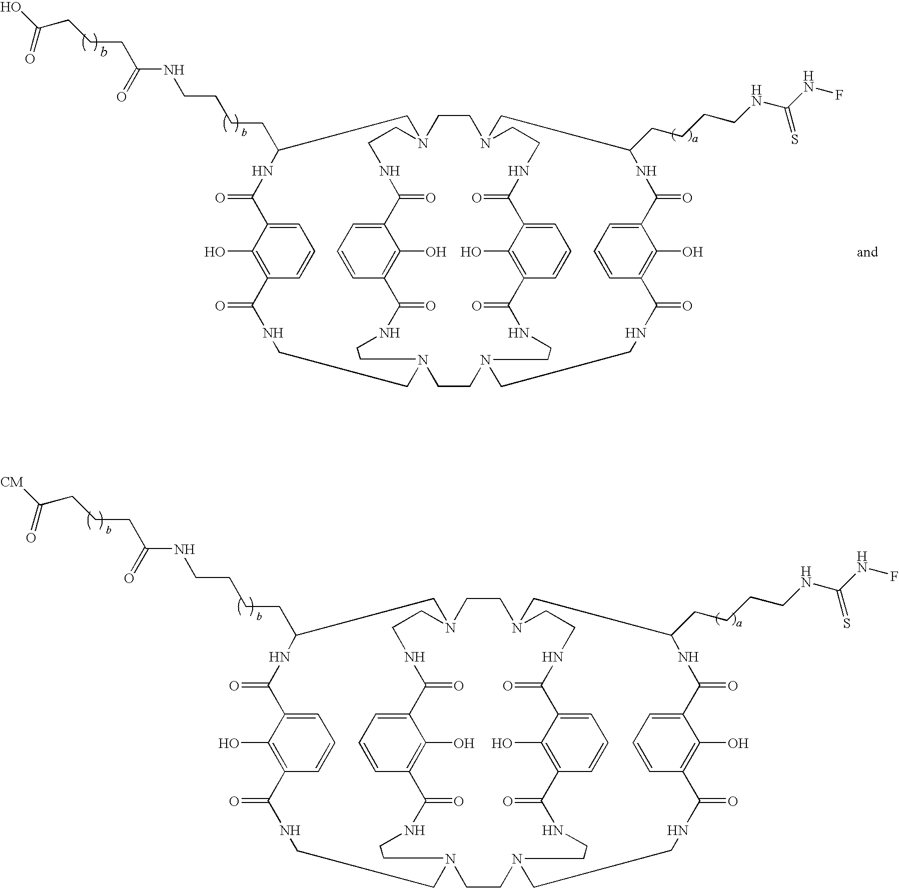

in which a and b are independently selected from integers greater than 0, e.g., integers from 0 to 15; and CM and F are as described above.

In still other exemplary embodiments, the functional moiety and the probe-linker are components of a structure bonded at the same point (e.g., the same atom) of the chelate, providing exemplary compounds having the formulae:

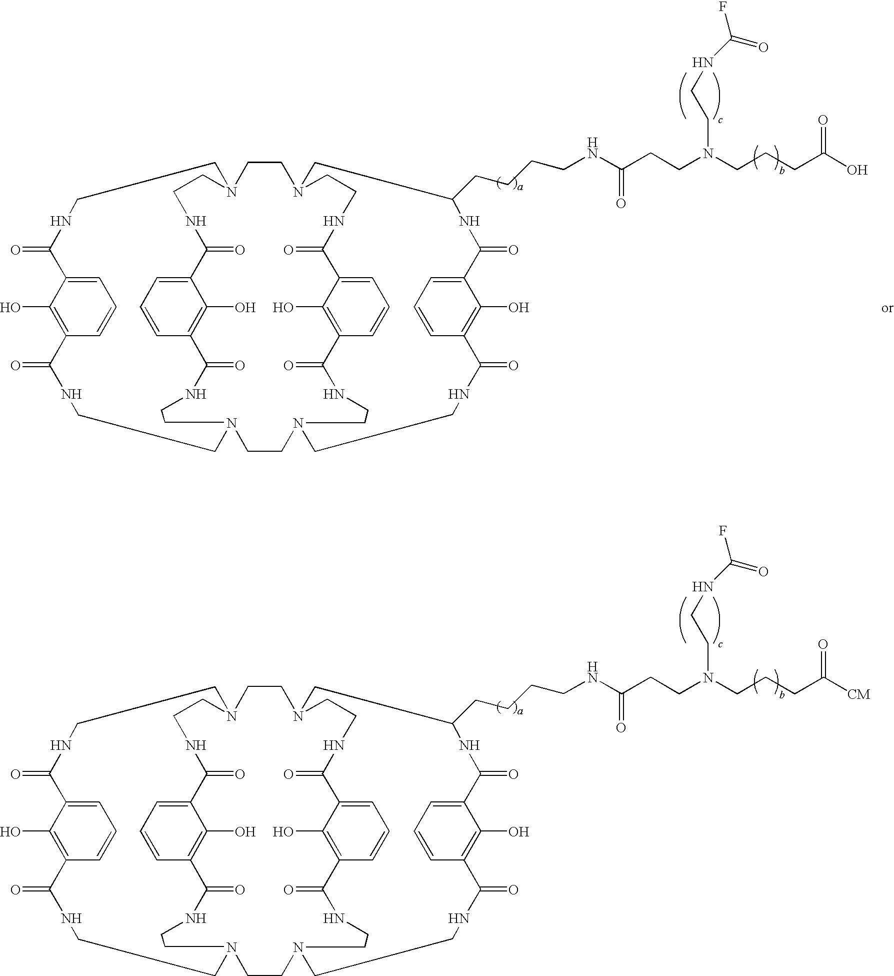

in which a, b and c are integers independently selected from integers greater than 0, e.g., integers from 0 to 15; CM is a carrier moiety (or solid support) and F is a probe.

The synthesis of the chelates disclosed herein, as well as other macrocyclic structures useful in the present invention can be found in US/2008/0213780.

Also provided are metal complexes formed from each of the above-described chelates of the invention. In an exemplary embodiment, the metal ion is selected to provide a metal chelate that is capable of transferring energy to the acceptor. Exemplary metal ions of use in to transfer energy to a fluorophore in compounds of this invention are lanthanide ions. Exemplary complexes are luminescent, and the metal ion is chosen according to meeting this criterion. In one exemplary embodiment, the metal is a member selected from the lanthanide group and the complex is preferably luminescent. Exemplary lanthanides include neodynium (Nd), samarium (Sm), europium (Eu), terbium (Tb), dysprosium (Dy) and ytterbium (Yb), of which europium and terbium are presently preferred.

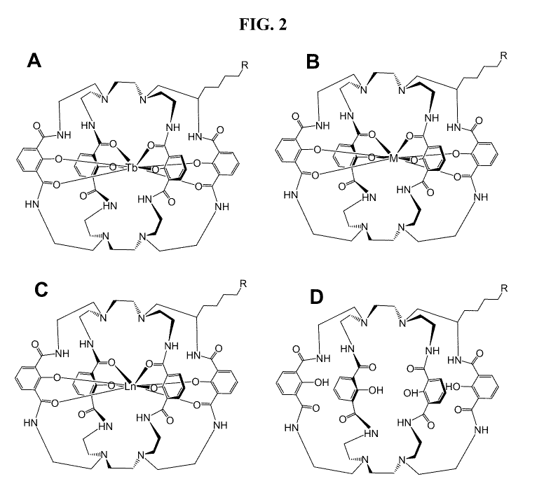

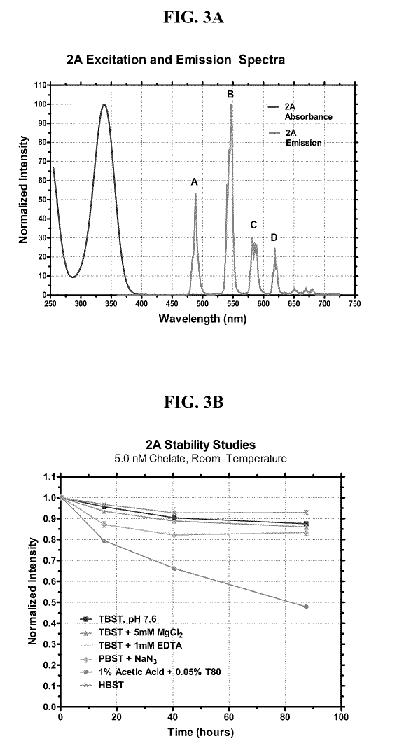

Structures of additional preferred energy transfer donors are shown in FIGS. 2A-D. The absorption spectrum of the compound of FIG. 2A is centered at approximately 340 nm and is quite broad allowing the use of photons over a wide energy range. The large Stokes Shift (the energy difference between excitation and emission wavelengths) is also important, since excitation energy can be easily filtered out and does not interfere complicate emission spectra. The four major emission peaks of the compound of FIG. 2A are centered at 490, 545, 570, and 620 nm allow significant multiplexing options through appropriately chosen acceptors that span the visible and near-IR ranges. Optimal FRET transfer usually occurs when the donor emission and acceptor excitation peaks have significant overlap, although it has been demonstrated that there is significant flexibility in these parameters. FRET coupling with the compound of FIG. 2A has the advantage of giving the acceptor a lifetime that is significantly extended relative to the conventional acceptor on its own. These properties combine in the present invention to allow the determination of multiple analytes in a single vial without interference or cross reaction.

The exceptional stability of the compounds of FIG. 2, such as 2A allows the present invention to be deployed in extreme environments such as high or low humidity, temperature and pressure as well as providing long shelf-life and limiting requirements for controlled storage.

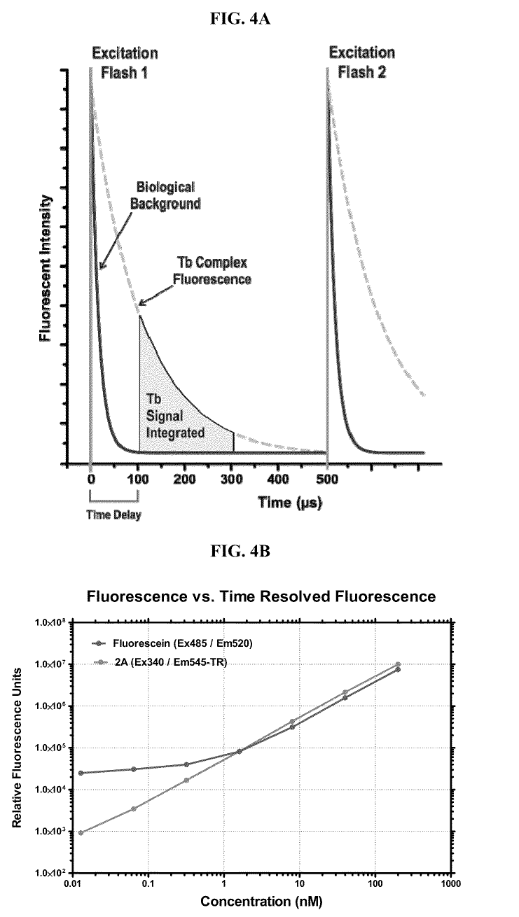

Luminescent lanthanide compounds are known to have exceptionally long lifetimes. While conventional organic dyes have lifetimes in the range of 1-10 nanoseconds, lanthanide based reporters have lifetimes of ˜1.2-2.5 ms. This unique property enables the time gated removal of background fluorescent interference (FIG. 4A). This property is especially useful in diagnostic applications based on the analysis of biological samples because it reduces or eliminates that need for sample preparation prior to analysis in a homogenous format. An excitation flash is delivered to a complex biological matrix followed by a delay to allow the interfering background fluorescence to dissipate. Finally, an integrated collection window of ˜50-2000 μs occurs yielding a signal almost free of interference. This effect greatly increases the low concentration reporter sensitivity as is demonstrated by comparing fluorescein under conventional settings with a compound of FIG. 2, with time-resolved settings (FIG. 4B).

Other reporter technologies that are preferred embodiments include the exchange of the energy transfer donor with a bioluminescence moiety resulting in Bioluminescent Resonance Energy Transfer (BRET). Typically this is achieved through the use of whole catalytic enzymes in the place of a conventional donor fluorophore. When substrate is added, bioluminescence is achieved at the site of the enzyme, which is imparted to an appropriate acceptor via resonant energy transfer. Additionally, technologies based on catalytic enzyme fragments may also form preferred embodiments of the current invention. Commercial examples include EMIT and CEDIA. This type of technology relies on the donor and acceptors being replaced by non-catalytic complimentary pieces of a catalytic enzyme. When the pieces are brought into close proximity, they form an active enzyme which can then turn over a reporter substrate generating a colorimetric or chemiluminescent (as examples) signal.

### Energy Transfer Acceptors

A variety of acceptors can be utilized in the invention. In exemplary embodiments, the energy transfer acceptor is a fluorophore, sometimes referred to as an acceptor fluorophore. Exemplary energy transfer acceptors of use in the invention are provided in Tables 1 and 2.

TABLE 1

| | Absorbance | Emission |
|---|---|---|
| Acceptor | (nm) | (nm) |
| Fluorescein (FITC) | 494 | 518 |
| Eosin | 524 | |
| TRITC | 543 | |
| Rhodamine 101 | 496 | 520 |
| Rhodamine Red | 570 | |
| Texas Red | 595 | 615 |
| Cy2 | 489 | 506 |
| Cy3 | 548 | 562 |
| Cy5 | 649 | 670 |
| Malachite Green | 630 | |
| Acridine orange | 500 | 530 |
| Bodipy 530/550 | 534 | 554 |
| YOYO-1 | 491 | 509 |
| YOYO-2 | 612 | 631 |
| Ca-Green | 506 | 534 |
| Ca-Orange | 555 | 576 |
| Ca-Crimson | 588 | 610 |
| Mg-Green | 506 | 532 |
| Na-Green | 507 | 532 |
| Fluorescein (FITC, FAM) | 494 | 518 |
| Eosin | 524 | |
| TET | 525 | 540 |
| HEX; JOE; VIC; CAL Fluor Orange 560 | 535 | 555 |
| ROX (5/6-carboxy Rhodamine); LC Red | 575 | 605 |
| 610; Cal Fluor Red 610 | | |
| Rhodamine 101 | 496 | 520 |
| Rhodamine Red | 570 | |
| Texas Red; LC Red 610; CAL | 590 | 610 |
| Fluor Red 610 | | |
| Cy2 | 489 | 506 |
| Cy3; NED; Quasar 570; Oyster 556 | 550 | 570 |
| Cy5; LC Red 670; Quasar 670; Oyster 645 | 649 | 670 |
| Tetramethyl Rhodamine (TAMRA, TMR, | 555 | 580 |
| TRITC) | | |
| Acridine orange | 500 | 530 |
| BODIPY TR-X | 588 | 616 |
| LC Red 640; Cal Fluor Red 635 | 625 | 640 |
| Nile Red | 485 | 525 |
| Oregon Green 488 | 493 | 520 |
| Oxonol V | 610 | 639 |

Protein Fluorophores

| Acceptor Name | Excitation (nm) | Emission (nm) |
|---|---|---|
| EGFP | 489 | 508 |
| dsRED | 558 | 583 |
| B-Phycoerythrin | 546, 565 | 575 |
| R-Phycoerythrin | 480, 546, 565 | 578 |
| allophycocyanin | 650 | 660 |

FRET Quenchers

| | | Absorption Max |
|---|---|---|
| Quencher Name | ε (cm⁻¹M⁻¹) | (nm) |
| QSY 7 | 90,000 | 570 |
| QSY-9 | 88,000 | 562 |
| QSY-35 | 23,000 | 475 |
| BHQ-1 | | 535 |
| BHQ-2 | | 580 |
| DDQ-I | | 430 |
| Dabcyl | | 475 |
| Eclipse | | 530 |
| Iowa Black FQ | | 532 |
| DDQ-II | | 630 |
| Iowa Black RQ | | 645 |

A wide selection of acceptors are available commercially, and are indicated in, though not limited to, Tables 1 and 2. FRET Quenchers may also be used in the current experimental format and will result in a negative read format test i.e. Increase analyte leads to reduced signal. The proprietary Alexfluor family is also indicated in Table 2 as an exemplary set of fluorophores that span the visible and NIR wavelengths providing many options for multiplexed tests.

TABLE 2
Alexa Fluor ® as Exemplary Acceptor Fluorophores
for Lumi4 ™-Tb Donor

| | | | Em | MW | |
|---|---|---|---|---|---|
| Fluorophore | Color¹ | Ex (nm) | (nm) | (g/mol) | ε (cm⁻¹M⁻¹) |
| Alexa Fluor 350 | blue | 346 | 442 | 410 | 19,000 |
| Alexa Fluor 405 | violet | 401 | 421 | 1028 | 34,000 |
| Alexa Fluor 430 | green | 434 | 541 | 702 | 16,000 |
| Alexa Fluor 488 | green | 495 | 519 | 643 | 71,000 |
| Alexa Fluor 500 | green | 502 | 525 | 700 | 71,000 |
| Alexa Fluor 514 | green | 517 | 542 | 714 | 80,000 |
| Alexa Fluor 532 | green | 532 | 554 | 721 | 81,000 |
| Alexa Fluor 546 | yellow-green | 556 | 573 | 1079 | 104,000 |
| Alexa Fluor 555 | green | 555 | 565 | ~1250 | 150,000 |
| Alexa Fluor 568 | orange | 578 | 603 | 792 | 91,300 |
| Alexa Fluor 594 | orange-red | 590 | 617 | 820 | 90,000 |
| Alexa Fluor 610 | red | 612 | 628 | 1172 | 138,000 |
| Alexa Fluor 633 | not vis | 632 | 647 | ~1200 | 100,000 |
| Alexa Fluor 647 | not vis | 650 | 665 | ~1300 | 239,000 |
| Alexa Fluor 660 | not vis | 663 | 690 | ~1100 | 132,000 |
| Alexa Fluor 680 | not vis | 679 | 702 | ~1150 | 184,000 |
| Alexa Fluor 700 | not vis | 702 | 723 | ~1400 | 192,000 |
| Alexa Fluor 750 | not vis | 749 | 775 | ~1300 | 240,000 |

In an exemplary embodiment of the present invention is the use of a peptide probe labeled with either a compound of FIG. 2, such as 2A, donor or an appropriately chosen acceptor. Many strategies exist for the labeling of a peptide terminus with a fluorophore including direct conjugation via N-terminal or C-terminal specific chemistries. Additionally, internal labeling may be achieved by modification of an amino acid side chain that occurs in mid-sequence. Finally, native molecular interactions may be leveraged to achieve a labeled peptide, such as using a biotin-labeled peptide and a streptavidin derivative labeled with either the donor or acceptor in an earlier reaction. Further variations on this mode of labeling may occur through the use of any known molecular interactions where one binding partner is associated with the peptide of interest and the binding partner is associated with the acceptor or donor of interest. These binding interactions may be through antibody recognition sequences such as multi-HIS, V5, c-Myc, FLAG tags or through more exotic binding interactions such as SNAP-tags, ACP tags, and CLIP tags that form covalent bonds following binding. These are exemplary indications of labeling strategies and are not intended to be limiting. It should be understood that there is no specific requirement for the location of donors and acceptors as described within this disclosure and that in all cases they are to be considered interchangeable.

### Kits

The present invention also provides kits for carrying out the invention. Kits may comprise a carrier, such as a box, carton, tube or the like, having in close confinement therein one or more containers, such as vials, tubes, ampoules, bottles, pouches, envelopes and the like. In various embodiments, the kits comprise one or more components selected from media, media ingredients and reagents for the detection of an analyte. For example, kits of the invention may comprise, in the same or different containers, a probe comprising one of an energy transfer pair (e.g., an energy transfer donor or an energy transfer acceptor) and a targeting moiety. The components may be contained within the same container or may be in separate containers to be admixed prior to use. The kits of the present invention may also comprise one or more instructions or protocols for carrying out a method of the present invention.

The kits may comprise any combination of probes disclosed herein. In one aspect, the invention provides a kit comprising a first probe comprising an energy transfer donor and a first targeting moiety; a second probe comprising an energy transfer acceptor and a second targeting moiety, wherein the first targeting moiety and the second targeting moiety have substantially the same structure; and instructions for using the first probe and the second probe to detect an analyte in a sample.

In one embodiment, the kit comprises a reaction vessel comprising an experimentally determined optimal ratio of a first probe comprising an energy transfer donor and a first targeting moiety and a second probe comprising an energy transfer acceptor and a second targeting moiety. In most cases it is expected that the stability of the probes will allow lyophilization of materials in the reaction vessel. In some cases, probes may not be stable to lyophilization and will be provided in a solubilized form. The exact formulation of the reagents in a kit may span some form of dry lyophilized or immobilized compounds to a liquid form that may contain buffering reagents, stabilizers preservatives in addition to the probes.

### Methods

The compounds and complexes of the invention are useful as probes in a variety of biological assay systems and diagnostic applications. In an exemplary embodiment, an energy transfer donor, such as the complex according to Formula I, is utilized in a procedure wherein emission from the complex excites at least one fluorophore in an assay. In another exemplary embodiment, emission from the complex excites at least two fluorophores in an assay, such that each fluorophore emits light of a characteristic wavelength and lifetime. In this example, each of the at least two fluorophores is distinguishable from the other on the basis of emission wavelength and/or lifetime. See Chen et al., J. Am. Chem. Soc., 122: 657-660 (2000). In a preferred embodiment, the complex of the invention distinguishably excites at least 2, 3, 4, 5, 6, 7, 8, 9 or at least 10 fluorophores essentially simultaneously.

The disclosed lanthanide complexes have particular utility in assays that are intended to detect or quantify binding or other modification of an assay component. These assays may incorporate one or more steps, including (a) contacting at least one member of a plurality of molecules with a binding partner capable of binding one of the molecules, (b) detecting a response indicative of the extent of binding between the at least one member of the plurality and the binding partner, and (c) correlating the response with the extent of binding or modification, or with a the activity of an enzyme that affects the modification. In some embodiments, the assays may include repeating the steps of contacting, detecting, and/or correlating for the same sample and/or a plurality of different samples. The assays may also involve providing a sample holder having a plurality of sample sites containing or supporting a corresponding plurality of samples, and sequentially and/or simultaneously repeating the steps of contacting, detecting, and/or correlating for the plurality of samples. The remainder of this section describes in more detail the steps of (a) contacting, (b) detecting, and (c) correlating.

The step of contacting assay components such as binding partners (e.g., nucleic acids, peptides, enzymes, enzyme modulators, substrates, products) with one another and/or with other species generally comprises any method for bringing any specified combination of these components into functional and/or reactive contact. A preferred method is by mixing and/or forming the materials in solution, although other methods, such as attaching one or more components (e.g., a complex according to Formula I, a species comprising a complex according to Formula I or other assay component) to a bead or surface, also may be used, as long as the components retain at least some function, specificity, and/or binding affinity following such attachment. The assay may be carried out in a device for manipulating fluids. Useful assay apparati having fluidics capability (e.g., microfluidics) suitable for contacting or otherwise preparing assay components are generally known in the art.

One or more of the assay components may comprise a sample, which typically takes the form of a solution containing one or more analyte that are biological and/or synthetic in origin. The sample may be a biological sample that is prepared from a blood sample, a urine sample, a swipe, or a smear, among others. Alternatively, the sample may be an environmental sample that is prepared from an air sample, a water sample, or a soil sample, among others. The sample typically is aqueous but may contain compatible organic solvents, buffering agents, inorganic salts, and/or other components known in the art for assay solutions.

The assay components and/or sample may be supported for contact and/or detection and/or analysis by any substrate or material capable of providing such support. Suitable substrates may include microplates, PCR plates, biochips, and hybridization chambers, among others, where features such as microplate wells and microarray (i.e., biochip) sites may comprise assay sites. Microplates may include 96, 384, 1536, or other numbers of wells. These microplates also may include wells having small (≈50 μL) volumes, elevated bottoms, and/or frusto-conical shapes capable of matching a sensed volume. Suitable PCR plates may include the same (or a similar) footprint, well spacing, and well shape as the preferred microplates, while possessing stiffness adequate for automated handling and thermal stability adequate for PCR. Suitable microarrays include nucleic acid and polypeptide microarrays, which are generally known in the art.

The step of detecting a response indicative of the extent of binding or modification generally comprises any method for effectuating such detection, including detecting and/or quantifying a change in, or an occurrence of, a suitable parameter and/or signal. The method may include luminescence and/or nonluminescence methods, and heterogeneous and/or homogeneous methods, among others.

Luminescence and nonluminescence methods may be distinguished by whether they involve detection of light emitted by a component of the sample. Luminescence assays involve detecting light emitted by a luminescent compound (or luminophore) and using properties of that light to understand properties of the compound and its environment. A typical luminescence assay may involve (1) exposing a sample to a condition capable of inducing luminescence from the sample, and (2) measuring a detectable luminescence response indicative of the extent of binding between the member of interest and a corresponding binding partner. Suitable luminescence assays include, among others, (1) luminescence intensity, which involves detection of the intensity of luminescence, (2) luminescence polarization, which involves detection of the polarization of light emitted in response to excitation by polarized light, (3) luminescence energy transfer, and (4) luminescence lifetime. A single assay mixture may be analyzed by one or more of these techniques. In a preferred embodiment, energy exchange between a luminescent complex of the invention and a fluorophore is utilized to detect the analyte (and optionally its degree of modification or binding to a binding partner) is utilized to determine both the emission wavelength and excitation lifetime of one or more fluorophores.

The detectable luminescence response generally comprises a change in, or an occurrence of, a luminescence signal that is detectable by direct visual observation and/or by suitable instrumentation. Typically, the detectable response is a change in a property of the luminescence, such as a change in the intensity, polarization, energy transfer, lifetime, and/or excitation or emission wavelength distribution of the luminescence. For example, energy transfer may be measured as a decrease in donor luminescence, an increase (often from zero) in acceptor luminescence, and/or a decrease in donor luminescence lifetime, among others. The detectable response may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence assays, the detectable response may be generated directly using a donor or acceptor associated with an assay component actually involved in binding, or indirectly using a donor or acceptor associated with another (e.g., reporter or indicator) component. Suitable methods and donors and acceptors for luminescently labeling assay components are described in the following materials, which are incorporated herein by reference: Richard P. Haugland, Handbook of Fluorescent Probes and Research Chemicals (6th ed. 1996).

Heterogeneous and homogeneous methods may be distinguished by whether they involve sample separation before detection. Heterogeneous methods generally require bulk separation of bound and unbound species. This separation may be accomplished, for example, by washing away any unbound species following capture of the bound species on a solid phase, such as a bead or microplate surface labeled with a trivalent metal or other suitable binding partner. Such metals may include gallium (Ga, including Ga(III)), iron (Fe), aluminum (Al), and/or zinc (Zn), among others. Suitable metals and other binding partners are described in more detail in US/2004/0249586, which is incorporated herein by reference. The extent of binding then can be determined directly by measuring the amount of captured bound species and/or indirectly by measuring the amount of uncaptured unbound species (if the total amount is known). Homogeneous methods, in contrast, generally do not require bulk separation but instead require a detectable response such as a luminescence response that is affected in some way by binding or unbinding of bound and unbound species without separating the bound and unbound species. Alternatively, or in addition, enzyme activity may result in increased or decreased energy transfer between a donor and acceptor of an energy transfer pair, based on whether the acceptor quenches or not, and based on whether enzyme activity in the assay results in increased or decreased proximity of the donor and acceptor. Homogeneous assays typically are simpler to perform but more complicated to develop than heterogeneous assays.

The step of correlating generally comprises any method for correlating the extent of binding with the extent of modification of the assay component being analyzed, and/or with the presence and/or activity of an enzyme that affects the modification. The nature of this step depends in part on whether the detectable response is simply detected or whether it is quantified. If the response is simply detected, it typically will be used to evaluate the presence of a component such as a substrate, product, and/or enzyme, or the presence of an activity such as an enzyme or modulator activity. In contrast, if the response is quantified, it typically will be used to evaluate the presence and/or quantity of a component such as a substrate, product, and/or enzyme, or the presence and/or activity of a component such as an enzyme or modulator.

The correlation generally may be performed by comparing the presence and/or magnitude of the response to another response (e.g., derived from a similar measurement of the same sample at a different time and/or another sample at any time) and/or a calibration standard (e.g., derived from a calibration curve, a calculation of an expected response, and/or a luminescent reference material). Thus, for example, in a energy transfer assay for cyclic nucleotide concentration, the cyclic nucleotide concentration in an unknown sample may be determined by matching the energy transfer efficiency measured for the unknown with the cyclic nucleotide concentration corresponding to that efficiency in a calibration curve generated under similar conditions by measuring energy transfer efficiency as a function of cyclic nucleotide concentration.

Thus, in one aspect, the invention provides a mixture of a complex of the invention and an analyte.

In another aspect, the invention provides a method of detecting the presence or absence of an analyte in a sample. The method comprises (a) contacting the sample and a composition of the invention; (b) exciting the complex; and (c) detecting luminescence from the complex. The presence or absence of the analyte can be indicated by the absence or presence of luminescence from the complex.

In an exemplary embodiment, the analyte, if present in said sample, competes with a probe molecule that includes a complex of the invention, for binding to a binding site located on a recognition molecule. In another exemplary embodiment, the analyte displaces the probe molecule from the binding site located on a recognition molecule, by binding to the binding site. In a further exemplary embodiment, the probe molecule is a complex of the invention.

Peptides doubly tagged with fluorescent dyes (Biophys. Chem., 1997, 67: 167-176) have previously been used as fluorogenic substrates for proteinases. In these assays dye-to-dye contact diminishes the fluorescence of the participating dyes by quenching. On enzymatic cleavage of the peptide link, the dye-tagged products dissociate, breaking dye to dye contact, thus relieving quenching of the fluorescence. To observe the increase in fluorescence indicative of enzyme activity usually requires breaking of a covalent bond in the linker. Fluorescent quenching has been used (Analytical Biochemistry, 1987, 165: 96-101) to measure the distance between a quencher and a fluorophore when attached to a peptide linker. Ai-Ping Wei et al (WO/1995/003429) use antibody-antigen reaction to break dye-to-dye contact in order that molecules in the dimer state (fluorescence quenched) become monomeric (fluorescence unquenched) to relieve quenching. This was used to form assays measuring specific antibodies to a recognized peptidic epitope that linked the two dyes. In common with many other homogeneous dequenching assays, while this method can measure antibodies specific to the epitope (used to bind the dyes) in a noncompetitive manner, its adaptation to measuring other analytes, possible only in competitive mode, suffers from disadvantage in that the fluorescence signal becomes indirectly proportional to analyte concentration.

Hence, in one aspect, the invention provides a kit including a recognition molecule and a compound or a complex of the invention. Exemplary recognition molecules include biomolecules, such as whole cells, cell-membrane preparations, antibodies, antibody fragments, proteins (e.g., cell-surface receptors, such as G-protein coupled receptors), protein domains, peptides, nucleic acids, and the like.

In one aspect, the invention provides a method of forming an assay complex comprising one or more probes as disclosed herein bound to a multivalent biomolecule. Any composition disclosed herein that may be considered to be an assay complex is one in which one or more probes, each comprising one of an energy transfer pair and a targeting moiety, are bound to a multivalent biomolecule such that energy transfer can occur. Thus, in one aspect, the invention provides a method of detecting an analyte in a sample comprising: (a) contacting the sample with one or more probes of the invention to form an assay complex comprising the one or more probes bound to the analyte; and (b) detecting the presence of the assay complex.

In one aspect, the invention provides a method of detecting an analyte in a sample comprising: (a) contacting the sample with (i) a first probe comprising an energy transfer donor and a first targeting moiety and (ii) a second probe comprising an energy transfer acceptor and a second targeting moiety to form an assay complex, wherein both the first probe and the second probe are bound to the analyte; and (b) detecting the presence of the assay complex.

In another aspect, the invention provides a method of detecting an analyte in a sample, comprising using a kit of the invention to form an assay complex by contacting the sample with a first probe comprising an energy transfer donor and a first targeting moiety; a second probe comprising an energy transfer acceptor and a second targeting moiety; wherein the assay complex comprises the first probe, the second probe and the analyte, wherein the both the first probe and the second probe are bound to the analyte; and (b) detecting the presence of the assay complex.

Use of the kit may comprise introducing a test sample into a reaction vessel, which typically will contain one or more probes disclosed herein. The sample may be any sample that may contain immunoreactive species and includes saliva, oral fluid, blood, serum, plasma, tears, or other bodily secretions. A brief incubation period occurs that allows association of the sample antibodies and the probes. This incubation may or may not require agitation of the sample. The required time for the reaction may have a wide range from assay to assay and will be determined individually for each reagent set. There is no need for the association reaction to reach equilibrium as results may be indicated relative to a negative control sample, internal control or other similarly effective means.

In one aspect, the invention provides a method of diagnosing a disease comprising: (a) contacting a sample comprising an analyte from a subject with one or more probes of the invention to form an assay complex comprising the one or more probes bound to the analyte; and (b) detecting the presence of the assay complex. Where the presence of the assay complex is ascertained, the disease state of the patient is accordingly determined. One of skill in the art can determine the appropriate analyte to be detected according to the type of disease being diagnosed and thus determine the appropriate probes for use. The disease being diagnosed can be any disease, but is particularly any disease disclosed herein.

In one embodiment, the detecting step of the methods described herein comprises detecting fluorescence resonance energy transfer (FRET) between the energy transfer donor and the energy transfer acceptor.

At a minimum the signal of the acceptor only will be read following excitation in a time-resolved format the specifics of which will be determined experimentally for each test. Additional readings may be taken at other control or multiplexing wavelengths.

### EXAMPLES

### Example 1

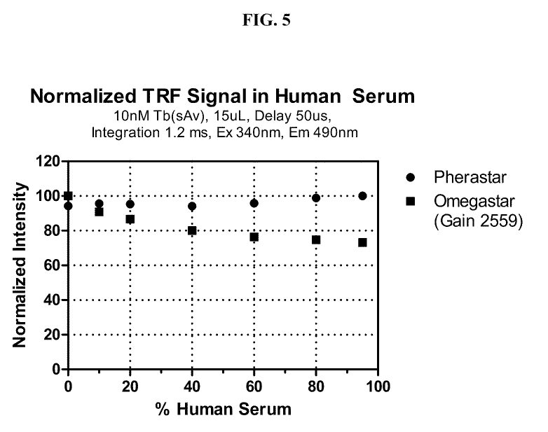

Streptavidin conjugated with Lumi4®-Tb (1.24 mg/mL, DOL=4.4) was diluted into TBST with varied amounts human serum. The pH was verified for all solutions to be ˜7.0 via pH paper. Identical plate and samples were read in both the Pherastar and Omegastar instruments (although any suitable reader may be used) (FIG. 5). Triplicate samples were read in a black 384 well plate with 15 μL sample volume.

On the Pherastar equipment there was little effect of serum concentration TRF signal. The slight increase in signal can be attributed to the interaction of high concentration serum proteins with the Tb Fluorophore. The Omegastar did exhibit decreasing signal intensity with increase serum concentration. This is due to a filter effect, where a less powerful excitation source in unable to excite all the Tb in the sample.

FRET transfer signal based on a single antibody binding two identical protein antigens that are differentially labeled with both a Lumi4®-Tb donor and an acceptor fluorophore was demonstrated. Limit of detection (LOD) and other results are presented only to complete the analysis but should not be considered a quantitative representation of the system capabilities.

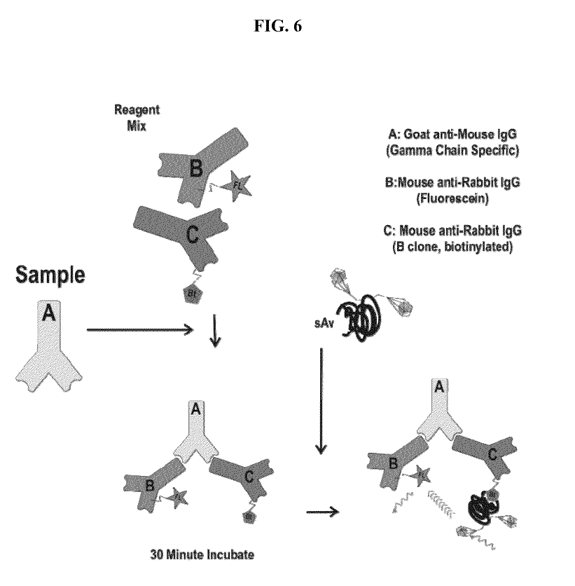

Goat anti-mouse IgG (A, Bridging Antibody, Unlabeled) was obtained from Southern Biotech (1030-01, Cross adsorption with Mouse IgM/IgA and human sera) and was used to represent the bridging sample antibody. The reagent antibodies were obtained from the same vendor and are the same antibody clone but labeled with fluorescein (B, 1030-02) or biotin (C, 1030-08). The experimental schematic is outlined below in FIG. 6.

Samples were prepared with 10 serially diluted (2×) solutions of Bridging Antibody A starting at ˜50 nM (7500 ng/mL) in TBST. Reagent antibodies B and C were combined in the reaction tube and represent the sample receiving matrix. The prepared samples were added to the reaction tube, vortexed and incubated for 30 minutes. Following incubation, the sAv-Tb reagent was added, and the samples were transferred in triplicate to a black 384 well plate. Samples were excited at 340 nm (Tb) and read at 520 nm (Fluorescein) following a 100 is delay, with a 400 is integration window and 500 flashes. It is expected that the 30 minute incubation step can be eliminated or at least reduced.

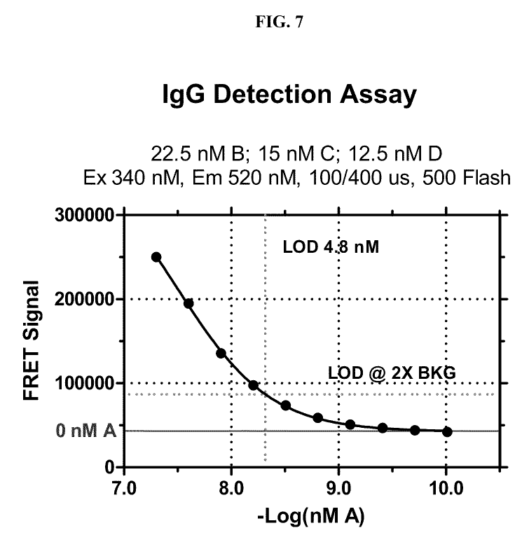

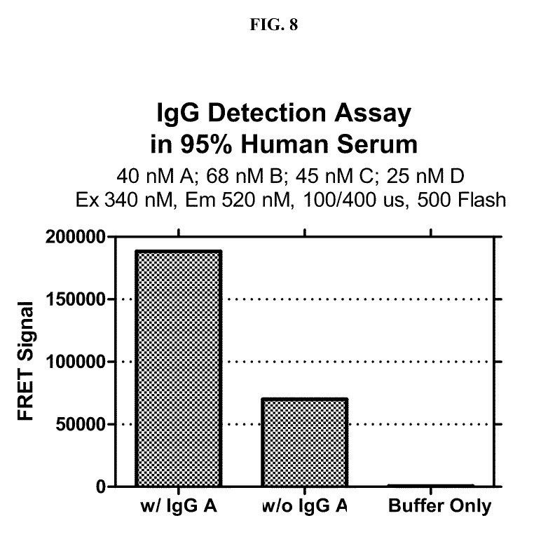

Data are presented in FIG. 7 showing the expected dose response curve. The upper saturation plateau region was not reached (where the bridging antibody is limiting the signal generation).

These data are promising with respect to the ability of a single antibody to bridge two differentially labeled but identical antigens resulting in a FRET signal. Current experiments indicate a LOD at 4.8 nM (720 ng/mL) for the bridging antibody.

Parameters for optimization include direct labeling of species B with the Lumi-4-Tb, relative concentrations of reactant, control of acceptor labeling level, incubation times, and removal of the sAv-Tb addition step.

A final experiment was performed to demonstrate the capabilities in human serum matrix. A sample was prepared in ˜95% human serum with and without the addition of the bridging antibody A (TBST was used in the w/o sample). Concentrations were modified slightly due to volume constraints, but experimental steps were as described previously.

The signal generated in the negative control sample is almost entirely due to predictable 520 nM signal contribution from the direct Tb luminescence and should not be considered non-specific signal arising from unexpected binding events in any appreciable amount. Experimental results of a TR-FRET based assay that relies on the presence of a bridging antibody in a patient serum sample are positive.

The articles “a,” “an” and “the” as used herein do not exclude a plural number of the referent, unless context clearly dictates otherwise. The conjunction “or” is not mutually exclusive, unless context clearly dictates otherwise. The term “include” is used to refer to non-exhaustive examples.

All publications, patents and patent applications cited herein are hereby incorporated by reference in their entirety for all purposes.

1. A composition comprising:
(a) a first probe comprising an energy transfer donor and a first targeting moiety;
(b) a second probe comprising an energy transfer acceptor and a second targeting moiety; and
(c) an antibody;
wherein the energy transfer donor comprises a macrocyclic moiety complexed to a lanthanide ion, the macrocyclic moiety having a structure according to Formula I: 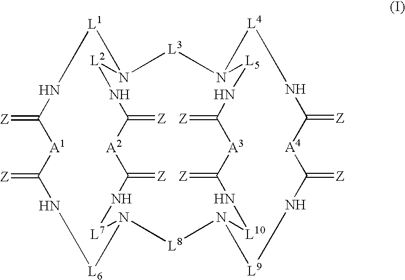
wherein each Z is a member independently selected from O and S;
L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
A¹, A², A³ and A⁴ are members independently selected from the general structure: 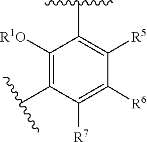
wherein each R¹ is a member independently selected from H, an enzymatically labile group, a hydrolytically labile group, a metabolically labile group and a single negative charge; and
each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂,
wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl;
R¹⁷ and R¹⁸ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and
R¹⁷ and R¹⁸, together with the atoms to which they are attached, are optionally joined to form a 5- to 7-membered ring.

2. The composition of claim 1 wherein the lanthanide ion is selected from the group consisting of samarium, dysprosium, europium and terbium.

3. The composition of claim 2 wherein the linker moieties L¹, L², L³, L⁴, L⁵, L⁶, L⁷, L⁸, L⁹ and L¹⁰ are members independently selected from substituted or unsubstituted C₁ to C₆ alkyl.

4. The composition of claim 3 wherein the linker moieties L¹, L², L³, L⁴, L⁵, L⁶, L⁷, L⁸, L⁹ and L¹⁰ are members independently selected from substituted or unsubstituted ethyl.

5. The composition of claim 4 wherein at least one of the linker moieties L¹, L², L³, L⁴, L⁵, L⁶, L⁷, L⁸, L⁹ and L¹⁰ is substituted by —(CH₂)₄R, wherein R is selected from a bond, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and wherein R links the donor to the first targeting compound.

6. The composition of claim 5 wherein the energy transfer donor has the structure: 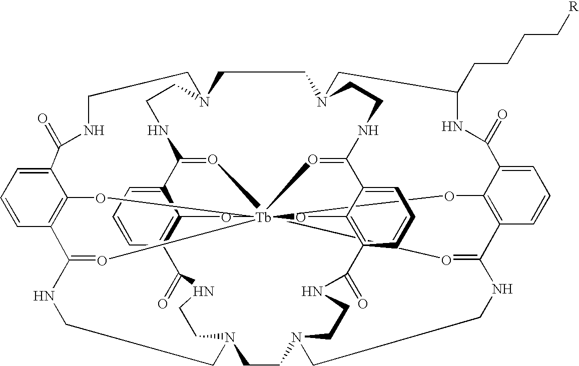
wherein R is selected from a bond, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl; and wherein R links the donor to the first targeting compound.

7. The composition of claim 1 wherein the energy transfer acceptor is a fluorophore.

8. The composition of claim 1 wherein the energy transfer acceptor is a dark quencher.

9. The composition of claim 1 wherein the first targeting moiety and the second targeting moiety have substantially the same structure.

10. The composition of claim 9 wherein the first targeting moiety and the second targeting moiety are each epitopes to the antibody.

11. The composition of claim 1 wherein the first probe and the second probe are independently selected from the group consisting of a polypeptide, a nucleic acid, a lipid, a polysaccharide and a small molecule.

12. The composition of claim 11 wherein at least one of the first probe and the second probe is a polypeptide.

13. The composition of claim 12 wherein the polypeptide comprises a sequence of a viral peptide.

14. The composition of claim 13 wherein the viral peptide is derived from the HIV I or II proteome.

15. The composition of claim 1 wherein the antibody is not bound to a solid support.

16. The composition of claim 1 wherein the first probe, the second probe or both are bound to the antibody.

17. A kit comprising:
(a) a first probe comprising an energy transfer donor and a first targeting moiety;
(b) a second probe comprising an energy transfer acceptor and a second targeting moiety, wherein the first targeting moiety and the second targeting moiety have substantially the same structure; and
(c) instructions for using the first probe and the second probe to detect an analyte in a sample.

18. The kit of claim 17 further comprising a reaction vessel comprising the first probe and the second probe.

19. The kit of claim 17 wherein the first probe and the second probe are lyophilized.

20. The kit of claim 17 wherein the energy transfer donor is a fluorophore.

21. The kit of claim 17 wherein the energy transfer donor has a structure according to Formula I above.

22. The kit of claim 17 wherein the energy transfer acceptor is a fluorophore.

23. The kit of claim 17 wherein the energy transfer acceptor is a dark quencher.

24. The kit of claim 17 wherein the first targeting moiety and the second targeting moiety are each epitopes to a same antibody.

25. A method of detecting an analyte in a sample, comprising:
(a) contacting the sample with (i) a first probe comprising an energy transfer donor and a first targeting moiety and (ii) a second probe comprising an energy transfer acceptor and a second targeting moiety to form the composition of claim 16 wherein the analyte is the antibody; and both the first probe and the second probe are bound to the antibody; and
(b) detecting the presence of the composition.

26. A method of detecting a first analyte and a second analyte in a sample, comprising:
(a) contacting the sample with (i) a first probe comprising a first energy transfer donor and a first targeting moiety; (ii) a second probe comprising a first energy transfer acceptor and a second targeting moiety; (iii) a third probe comprising a second energy transfer donor and a third targeting moiety; and (iv) a fourth probe comprising a second energy transfer acceptor and a fourth targeting moiety to form a first assay complex and a second assay complex
wherein the first assay complex comprises a first multivalent biomolecule bound to the first probe and to the second probe;
the second assay complex comprises a second multivalent biomolecule bound to the third probe and to the fourth probe;
the first targeting moiety and the second targeting moiety have substantially the same structure; and the third targeting moiety and the fourth targeting moiety have substantially the same structure; and
the first analyte is the first multivalent biomolecule and the second analyte is the second multivalent biomolecule; and
(b) detecting the presence of the first assay complex and the second assay complex.

27. The method of claim 26 wherein the first targeting moiety and the second targeting moiety are each epitopes to a first antibody; and the third targeting moiety and the fourth targeting moiety are each epitopes to a second antibody.

28. The method of claim 26 wherein the first analyte is a first antibody and the second analyte is a second antibody.

29. A method of detecting an analyte in a sample comprising:
(a) using the kit of claim 17 to form an assay complex by contacting the sample with the first probe and the second probe, wherein the assay complex comprises the first probe, the second probe and the analyte, wherein both the first probe and the second probe are bound to the analyte; and
(b) detecting the presence of the assay complex.

30. The method of claim 29 wherein the detecting step comprises detecting fluorescence resonance energy transfer (FRET) between the energy transfer donor and the energy transfer acceptor.
